(19) United States
(12) Patent Application Publication
(10) Pub. No.: US 2017/0028007 A1
(43) Pub. Date: Feb. 2, 2017

(54) SOLID DISPERSION CONTAINING DESMODIUM STYRACIFOLIUM (OSB.) MERR. FLAVONOIDS, METHOD OF PREPARING SAME, AND USE THEREOF

(71) Applicant: HUMANWELL HEALTHCARE (GROUP) CO., LTD., Wuhan (CN)

(72) Inventors: XUEHAI WANG, Wuhan (CN); LI'E LI, Wuhan (CN); YONG XU, Wuhan (CN); JIE LI, Wuhan (CN); RONGHUA TU, Wuhan (CN); GANG CHEN, Wuhan (CN); RUBIN CAO, Wuhan (CN); YUN FENG, Wuhan (CN); ZHONGWEN YANG, Wuhan (CN); ZHAOZE FAN, Wuhan (CN); YANPING YU, Wuhan (CN); QIANG XIAO, Wuhan (CN); LU HUANG, Wuhan (CN); CHENGBING YANG, Wuhan (CN); TIANCI HUANG, Wuhan (CN); HUA TIAN, Wuhan (CN); JING YANG, Wuhan (CN)

(21) Appl. No.: 15/101,928

(22) PCT Filed: Jul. 15, 2014
(86) PCT No.: PCT/CN2014/082250

§ 371 Date: Jun. 5, 2016

(30) Foreign Application Priority Data
Dec. 5, 2013 (CN) 201310643153.3

(51) Int. Cl. A61K 36/48; A61K 9/20; A61K 9/48

(52) U.S. Cl. CPC A61K 36/48; A61K 9/4833; A61K 9/2095

(57) ABSTRACT

A solid dispersion containing Desmodium Styracifolium (Osb.) Merr. flavonoids, method of preparing same and use thereof. The Desmodium Styracifolium (Osb.) Merr. flavonoids are alcohol extract of Desmodium Styracifolium (Osb.) Merr., and the solid dispersion is used for preparing a drug for treating urinary tract calculi.

### FIELD

The present disclosure relates to the field of pharmaceutical formulation, and particularly to an oral solid dispersion containing total flavonoids of Desmodium Styracifolium and use thereof. More particularly, the present disclosure relates to a solid dispersion containing total flavonoids of Desmodium Styracifolium as an active ingredient, a solid dispersion formulation containing total flavonoids of Desmodium Styracifolium and a preparation method thereof, and uses of the solid dispersion and the solid dispersion formulation containing total flavonoids of Desmodium Styracifolium in the preparation of a medicament for treating urinary stone.

### BACKGROUND

Desmodium Styracifolium is a dried overground part of leguminous plants, Desmodium styracifolium (Osb.) Merr., as a traditional Chinese medicine recorded in Part I of Chinese Pharmacopoeia (2010 edition) having efficacy in disinhibiting dampness-abating jaundice and disinhibiting urine and freeing strangury. A prescription preparation of stranguria-treating and calculus-removing tablet containing Desmodium Styracifolium as its essential ingredient, also recorded in Chinese Pharmacopoeia, can be used to treat bladder dampness-heat, stone strangury with roughness and pain in the urethra, lithangiuria and urinary infection belonging to dampness and heat in liver, gallbladder and urinary bladder. However, the raw material of the stranguria-treating and calculus-removing tablet is a crude extract of the Desmodium Styracifolium which is prepared by a traditional water-extraction and alcohol-precipitation extraction method, and this tablet also has a plurality of drawbacks, such as unclear effective components in Chinese herb, overdose in clinic (6 times a day, three pills one time, sugar-coated tablets or film-coated tablets, each pill containing 0.12 g dry extract) and inadequate standard in quality control. The stone discharging agent, such as potassium citrate, thiazide diuretic, magnesium agent, and acetyl cysteine, which is often used in clinic to treating the lithangiuria, with an non-ideal efficacy and significant toxicity and side effect. Chinese patent medicine, such as “Mi Shi Tong”, lithagogue infusion, and stranguria-treating and calculus-removing tablets, is commonly used medicaments with exact effect. However, similar with the stranguria-treating and calculus-removing tablets, all these traditional Chinese medicines still exist such problems, such as original pharmaceutical process, difficulties in quality control, inaccurate quantitative detection method, and overdose, that there is a relative great distance as compared with international standards and does not meet the requirements of modern clinical medicine, in addition, the total flavonoids of Desmodium Styracifolium is a water-insoluble medicament.

Therefore, it needs to research and develop new traditional Chinese medicines related to total flavonoids of Desmodium Styracifolium with safety and efficacy, controllable quality, and high dissolution rate. Current research on formulations related to total flavonoids of Desmodium Styracifolium still needs to be strengthened. It is very necessary to provide an oral solid formulation of the total flavonoids of Desmodium Styracifolium with a superior therapeutic effect than that of the existing medicaments, stable and controllable quality for clinic, better absorption in vivo, safety and efficacy, and economic.

### SUMMARY

The present disclosure is accomplished based on the following discoveries. The chemical composition of Desmodium styracifolium contains flavonoids, alkaloids, phenols, tannins and polysaccharides, in which the total flavonoids are the main effective component. Pharmacology experiments show that the active ingredient (effective component) of Desmodium styracifolium, i.e., the total flavonoids of Desmodium styracifolium, has significant pharmacological functions of dissolving stone, discharging stone and reducing the formation of stones. In addition, the inventor has found that, total flavonoid of Desmodium Styracifolium is almost insoluble in water, methanol, ethanol, 0.1 mol/L HCl solution, ethyl acetate, chloroform and acetone, but is soluble in 25% ethanol, and is freely soluble in 0.1 mol/L NaOH solution, dimethylsulfoxide and 50% ethanol. Total flavonoid of Desmodium Styracifolium is almost insoluble in water due to the physical and chemical properties of its active pharmaceutical ingredients (APIs), but its extractum or powders is prone to sticky (increasing viscosity and easy to agglomerate). Therefore, the total flavonoid of Desmodium Styracifolium will has a very low dissolution rate if formulated into common pharmaceutical formulations. Thus, granulating effect is very poor and granulation almost cannot be carried out using conventional manufacturing processes, which easily leads to the oral solid formulation containing total flavonoid of Desmodium Styracifolium thus obtained having a plurality of drawbacks, such as slow dissolution, unstable product quality, low bioavailability, and low clinical efficacy. Additionally, the absorption of medicaments is based on its dissolution, its bioavailability in vivo is relate to the dissolution rate in vitro to a certain degree, therefore, the dissolution rate of a medicament plays a direct role on its absorption, the dissolution of the medicament in a formulation is a step to control whether the medicament can exert its efficacy. Furthermore, enhancing the dissolution and release of the medicament is most critical to improve the absorption rate of oral formulations in human body, which will increase the maximum plasma concentration of the active ingredient after 3 hours from oral administration

The present application achieves beneficial effects throughout a large amount of researches and experiments. During the study, it has been surprisingly found that, dispersing the total flavonoid of Desmodium Styracifolium into a composition of hydrophilic solid dispersion carrier materials at a certain ratio via a solid dispersion technology improves the wettability of the drug, ensures its high dispersibility and thus contributes to the dissolution and absorption of drugs. Poorly soluble drugs may achieve a significantly increased solubility, a more effectively reduced surface contact angle, an improved wettability, thus an increased solubility by adding a surfactant into the carrier material

Therefore, the present disclosure provides a solid dispersion containing total flavonoids of Desmodium Styracifolium as an active ingredient, a tablet containing total flavonoids of Desmodium Styracifolium and a preparation method thereof, and use of the solid dispersion and the tablet containing total flavonoids of Desmodium Styracifolium in the preparation of a medicament for treating urinary stone, on the basis of the existing pharmaceutically acceptable adjuvant and production conditions under the premise of low production costs, simple and practicable manufacturing processes, and suitable for large scale industrial production. An oral solid formulation obtained according to the present disclosure has good bioavailability and drug stability.

In a first aspect of the present disclosure, total flavonoids of Desmodium Styracifolium are provided. In an embodiment of the present disclosure, the total flavonoids of Desmodium Styracifolium are alcohol extract of Desmodium Styracifolium.

According to an embodiment of the present disclosure, a solid dispersion is provided, which contains total flavonoids of Desmodium Styracifolium provided in a form of the alcohol extract of Desmodium Styracifolium as an active ingredient. In an embodiment of the present disclosure, the alcohol extract of Desmodium Styracifolium is obtained by the following steps: heating and refluxing a raw material of Desmodium Styracifolium for extraction with ethanol having a concentration ranging from 50% to 95% and a weight ranging from 8 to 14 times as heavy as the raw material, so as to obtain an extracting solution of Desmodium Styracifolium; concentrating the extracting solution of Desmodium Styracifolium, so as to remove ethanol; and subjecting the extracting solution of Desmodium Styracifolium after concentrated to adsorption onto a macroporous resin column, so as to obtain the alcohol extract of Desmodium Styracifolium. In an embodiment of the present disclosure, the extracting solution of Desmodium Styracifolium is obtained by: heating and refluxing the raw material of Desmodium Styracifolium for extraction, 1 to 3 times with 1 to 3 hours for each time, with ethanol having the concentration ranging from 50% to 95% and the weight ranging from 8 to 14 as heavy as the raw material, to obtain alcohol extracting solutions of Desmodium styracifolium, and mixing the ethanol extracting solutions.

The present invertor has found that, an extract product of total flavonoids of Desmodium Styracifolium has a content (in a dry product, %) of total flavonoids of Desmodium Styracifolium ranging from 50% to 80% in which a content (in a dry product, %) of schaftoside is from 3.0% to 12.0%.

Specifically, in some embodiments of the present disclosure, a method of the present disclosure for preparing the total flavonoids of Desmodium Styracifolium may include the following steps:

weighing a raw material of Desmodium Styracifolium, heating and refluxing the raw material for extraction, 1 to 3 times with 1 to 3 hours for each time, at a temperature of 50° C. to 60° C. with ethanol having a concentration of 50% to 95% and a weight ranging from 8 to 14 times as heavy as the raw material, so as to obtain alcohol extracting solutions of Desmodium Styracifolium followed by mixing;

concentrating the alcohol extracting solution to be of a volume 2 to 8 times the weight of the raw material followed by still standing and filtering to obtain a filtrate;

subjecting the filtrate to adsorption onto an AB-8 macroporous resin column at a flow rate ranging from 1 to 3 column bed volumes per hour, eluting and purifying with water having a volume ranging from 8 to 12 times the weight of filled resin, and eluting with ethanol having a concentration of 40% to 95% and a volume ranging from 6 to 10 column bed volumes at a flow rate ranging from 2 to 4 column bed volumes per hour, to obtain an eluted solution; and

concentrating the eluted solution to recycle ethanol and to obtain a concentrated solution with a relative density ranging from 1.10 to 1.30 followed by drying and then smashing, so as to obtain an extract product of total flavonoids of Desmodium Styracifolium with a content of the total flavonoids of Desmodium Styracifolium reaching 50% to 80%, in which a content of schaftoside is from 3.0% to 12.0%. The extract after drying is collected, sealed, weighted and stored in a dry place.

Unless specifically stated, a concentration of ethanol described in the present disclosure refers to a volume fraction (V/V) of ethanol contained in 100 mL volume of ethanol solution.

Specifically, according to some embodiments of the present disclosure, the preparation process and the technology parameters for the total flavonoids of Desmodium Styracifolium are investigated and studied in detail, resulting in a preferable condition, which is verified in a pilot test and successfully transited into industrial production.

Specifically, in an embodiment of the present disclosure, a method for preparing the total flavonoids of Desmodium Styracifolium may include the following steps:

weighing a raw material of Desmodium Styracifolium, heating and refluxing at a temperature of 55° C. for 2 hours for first extraction with ethanol having a concentration of 80% and a weight 12 times as heavy as the raw material, heating and refluxing at a temperature of 55° C. for 1.5 hours for second extraction with ethanol having a concentration of 80% and a weight 10 times as heavy as the raw material, so as to obtain alcohol extracting solutions of Desmodium Styracifolium followed by mixing;

concentrating the alcohol extracting solution to be of a volume 5 times the weight of the raw material followed by still standing and filtering, to obtain a filtrate;

subjecting the filtrate to adsorption onto an AB-8 macroporous resin column at a flow rate of 2 column bed volumes per hour, eluting and purifying with water having a volume 10 times the weight of filled resin, and eluting with ethanol having a concentration of 60% and a volume of 8 column bed volumes at a flow rate of 2 column bed volumes per hour, to obtain an eluted solution; and

concentrating the eluted solution to recycle ethanol and to obtain a concentrated solution with a relative density of 1.22, followed by drying under reduced pressure at a temperature of 75° C. and then smashing to obtain total flavonoids of Desmodium Styracifolium.

According to an embodiment of the present disclosure, contents of effective ingredients and effective substances in Desmodium Styracifolium drugs have been increased. A content of total flavonoids of Desmodium Styracifolium is between 50% and 80% (by the extract after dried, %), in which a content of schaftoside is between 3.0% and 12.0% (by the extract after dried, %).

The solid dispersion technology described in the present disclosure refers to a technology for forming a system in which solid dispersions, i.e. drugs, is uniformly dispersed into a certain solid carrier in a state of particle, microcrystalline or molecular. Water-soluble and high hydrophilic substance often used as a solid dispersion carrier to increase the solubility and dissolution rate of some poorly soluble drugs, and increase the bioavailability of the drug after oral administration. A dispersion state of the drug in a carrier includes a simple eutectic mixture, a solid solution, a partial crystalline, a glassy solid solution and a molecular complexes, etc.

In an embodiment of the present disclosure, the solid dispersion is provided in an oral solid formulation form of a capsule, granules or a tablet. Alternatively, the tablet is a sugar-coated tablet, a film-coated tablet, a dispersible tablet, a sustained-release tablet, or a controlled-release tablet. The dissolution rates of all these oral solid formulations of total flavonoids of Desmodium Styracifolium have been improved significantly. Alternatively, the oral solid formulation form of total flavonoids of Desmodium Styracifolium is a capsule or a tablet containing solid dispersion of total flavonoids of Desmodium Styracifolium.

In an embodiment of the present disclosure, the oral solid formulation containing the solid dispersion of total flavonoids of Desmodium Styracifolium (herein, “oral solid formulation containing the solid dispersion of total flavonoids of Desmodium Styracifolium” is interchangeable with “oral solid formulation containing total flavonoids of Desmodium Styracifolium” and “solid dispersion formulation containing total flavonoids of Desmodium Styracifolium”) includes: total flavonoids of Desmodium Styracifolium and a pharmaceutical adjuvant. The total flavonoids of Desmodium Styracifolium are alcohol extract of Desmodium Styracifolium obtained according to the method for preparing the total flavonoids of Desmodium Styracifolium of the present disclosure, and the pharmaceutical adjuvant includes at least one of a solid dispersion carrier and a surfactant.

In an embodiment of the present disclosure, the capsule containing solid dispersion of total flavonoids of Desmodium Styracifolium (herein, “capsule containing solid dispersion of total flavonoids of Desmodium Styracifolium” is interchangeable with “capsule containing total flavonoids of Desmodium Styracifolium”) includes: total flavonoids of Desmodium Styracifolium and a pharmaceutical adjuvant. The total flavonoids of Desmodium Styracifolium are alcohol extract of Desmodium Styracifolium obtained according to the method for preparing the total flavonoids of Desmodium Styracifolium of the present disclosure, and the pharmaceutical adjuvant includes at least one of a solid dispersion carrier and a surfactant.

In an embodiment of the present disclosure, the tablet containing solid dispersion of total flavonoids of Desmodium Styracifolium (herein, “tablet containing solid dispersion of total flavonoids of Desmodium Styracifolium” is interchangeable with “tablet containing total flavonoids of Desmodium Styracifolium”) includes: total flavonoids of Desmodium Styracifolium and a pharmaceutical adjuvant. The total flavonoids of Desmodium Styracifolium are alcohol extract of Desmodium Styracifolium obtained according to the method for preparing the total flavonoids of Desmodium Styracifolium of the present disclosure, and the pharmaceutical adjuvant includes at least one of a solid dispersion carrier and a surfactant.

In an embodiment of the present disclosure, the oral solid formulation containing the solid dispersion of total flavonoids of Desmodium Styracifolium may include: 33 to 133 weight parts of total flavonoids of Desmodium Styracifolium, 198 to 798 weight parts of the solid dispersion carrier, 6.6 to 133 weight parts of the surfactant, 1 to 50 weight parts of filler, 1 to 50 weight parts of disintegrant, and 1 to 10 weight parts of lubricant.

In some embodiments of the present disclosure, the solid dispersion carrier in the oral solid formulation containing the solid dispersion of total flavonoids of Desmodium Styracifolium is a water-soluble solid dispersion carrier material, which is at least one selected from povidone K₃₀, polyethylene glycol 2000, polyethylene glycol 4000, polyethylene glycol 6000, citric acid, succinic acid, dextran, galactose, saccharose, glucose, modified starch, microcrystalline cellulose, poloxamer 188, and D-mannitol, the solid dispersion carrier alternatively is at least one of povidone K₃₀, polyethylene glycol 6000, D-mannitol, and poloxamer 188, and alternatively, is at least one of povidone K₃₀, and poloxamer 188.

In some embodiments of the present disclosure, the surfactant in the oral solid formulation containing the solid dispersion of total flavonoids of Desmodium Styracifolium is at least one selected from coconut oil amine polyglycol ether, glycerol polyoxyethylene ether, Tween 20, Tween 40, Tween 60, Tween 80, Myrj 40, Brij 30, methoxypolyethylene glycol, and sodium dodecyl sulfate, alternatively, the surfactant is at least one of Tween 80, methoxypolyethylene glycol, and sodium dodecyl sulfate, and alternatively, is sodium dodecyl sulfate.

In some embodiments of the present disclosure, the filler in the oral solid formulation containing the solid dispersion of total flavonoids of Desmodium Styracifolium is at least one selected from corn starch, dextrin, lactose, pregelatinized starch, saccharose, microcrystalline cellulose, mannitol, sorbitol, xylitol, calcium hydrophosphate, and calcium carbonate, alternatively, the filler is at least one of microcrystalline cellulose, lactose, pregelatinized starch, and alternatively, is lactose.

In some embodiments of the present disclosure, the disintegrant in the oral solid formulation containing the solid dispersion of total flavonoids of Desmodium Styracifolium is at least one selected from sodium carboxymethyl starch, low-substituted hydroxypropyl cellulose (L-HPC), cross-linked povidone, dry starch, sodium croscarmellose, and carboxymethyl cellulose calcium, alternatively, the disintegrant is one of sodium croscarmellose, cross-linked povidone, and sodium carboxymethyl starch, and alternatively, is sodium croscarmellose.

In some embodiments of the present disclosure, the lubricant in The oral solid formulation containing total flavonoids of Desmodium Styracifolium is at least one selected from magnesium stearate, talc, aerosil, magnesium dodecyl sulfate, sodium dodecyl sulfate, sodium benzoate, and sodium stearyl fumarate, alternatively, the lubricant is one of magnesium stearate, aerosil, and sodium stearyl fumarate, and alternatively, is sodium stearyl fumarate.

In a specific embodiment of the present disclosure, the oral solid formulation containing the solid dispersion of total flavonoids of Desmodium Styracifolium may include: 33 weight parts of total flavonoids of Desmodium Styracifolium, 132 weight parts of povidone K₃₀, 66 weight parts of poloxamer 188, 6.6 weight parts of sodium dodecyl sulfate, 50 weight parts of lactose, 20 weight parts of cross-linked povidone, and 1 weight part of aerosil.

In a specific embodiment of the present disclosure, the oral solid formulation containing the solid dispersion of total flavonoids of Desmodium Styracifolium may include: 33 weight parts of total flavonoids of Desmodium Styracifolium, 132 weight parts of povidone K₃₀, 66 weight parts of poloxamer 188, 19.8 weight parts of sodium dodecyl sulfate, 30 weight parts of lactose, 20 weight parts of sodium croscarmellose, and 3 weight parts of sodium stearyl fumarate.

In a specific embodiment of the present disclosure, the oral solid formulation containing the solid dispersion of total flavonoids of Desmodium Styracifolium may include: 33 weight parts of total flavonoids of Desmodium Styracifolium, 132 weight parts of povidone K₃₀, 66 weight parts of poloxamer 188, 33 weight parts of sodium dodecyl sulfate, 10 weight parts of microcrystalline cellulose, 20 weight parts of sodium croscarmellose, and 2 weight parts of magnesium stearate.

In a specific embodiment of the present disclosure, the oral solid formulation containing the solid dispersion of total flavonoids of Desmodium Styracifolium may include: 33 weight parts of total flavonoids of Desmodium Styracifolium, 132 weight parts of povidone K₃₀, 66 weight parts of poloxamer 188, 19.8 weight parts of Tween 80, 40 weight parts of lactose, 5 weight parts of sodium carboxymethyl starch, and 4 weight parts of sodium stearyl fumarate.

In a specific embodiment of the present disclosure, the oral solid formulation containing the solid dispersion of total flavonoids of Desmodium Styracifolium may include: 33 weight parts of total flavonoids of Desmodium Styracifolium, 132 weight parts of povidone K₃₀, 66 weight parts of poloxamer 188, 19.8 weight parts of methoxypolyethylene glycol, 20 weight parts of microcrystalline cellulose, 15 weight parts of sodium carboxymethyl starch, and 3 weight parts of sodium stearyl fumarate.

In a specific embodiment of the present disclosure, the oral solid formulation containing the solid dispersion of total flavonoids of Desmodium Styracifolium may include: 50 weight parts of total flavonoids of Desmodium Styracifolium, 200 weight parts of povidone K₃₀, 100 weight parts of poloxamer 188, 10 weight parts of sodium dodecyl sulfate, 50 weight parts of lactose, 20 weight parts of sodium croscarmellose, and 5 weight parts of sodium stearyl fumarate.

In a specific embodiment of the present disclosure, the oral solid formulation containing the solid dispersion of total flavonoids of Desmodium Styracifolium may include: 50 weight parts of total flavonoids of Desmodium Styracifolium, 200 weight parts of povidone K₃₀, 100 weight parts of poloxamer 188, 30 weight parts of sodium dodecyl sulfate, 50 weight parts of lactose, 20 weight parts of sodium croscarmellose, and 5 weight parts of sodium stearyl fumarate.

In a specific embodiment of the present disclosure, the oral solid formulation containing the solid dispersion of total flavonoids of Desmodium Styracifolium may include: 50 weight parts of total flavonoids of Desmodium Styracifolium, 200 weight parts of povidone K₃₀, 100 weight parts of poloxamer 188, 50 weight parts of sodium dodecyl sulfate, 50 weight parts of lactose, 20 weight parts of sodium croscarmellose, and 5 weight parts of sodium stearyl fumarate.

In a specific embodiment of the present disclosure, the oral solid formulation containing the solid dispersion of total flavonoids of Desmodium Styracifolium may include: 50 weight parts of total flavonoids of Desmodium Styracifolium, 200 weight parts of povidone K₃₀, 100 weight parts of poloxamer 188, 30 weight parts of Tween 80, 50 weight parts of lactose, 20 weight parts of sodium croscarmellose, and 5 weight parts of sodium stearyl fumarate.

In a specific embodiment of the present disclosure, the oral solid formulation containing the solid dispersion of total flavonoids of Desmodium Styracifolium may include: 50 weight parts of total flavonoids of Desmodium Styracifolium, 200 weight parts of povidone K₃₀, 100 weight parts of poloxamer 188, 30 weight parts of methoxypolyethylene glycol, 50 weight parts of lactose, 20 weight parts of sodium croscarmellose, and 5 weight parts of sodium stearyl fumarate.

In a specific embodiment of the present disclosure, the oral solid formulation containing the solid dispersion of total flavonoids of Desmodium Styracifolium may include: 50 weight parts of total flavonoids of Desmodium Styracifolium, 200 weight parts of povidone K₃₀, 100 weight parts of poloxamer 188, 30 weight parts of sodium dodecyl sulfate, 50 weight parts of pregelatinized starch, 20 weight parts of sodium croscarmellose, and 5 weight parts of sodium stearyl fumarate.

In a specific embodiment of the present disclosure, the oral solid formulation containing the solid dispersion of total flavonoids of Desmodium Styracifolium may include: 50 weight parts of total flavonoids of Desmodium Styracifolium, 200 weight parts of povidone K₃₀, 100 weight parts of poloxamer 188, 50 weight parts of sodium dodecyl sulfate, 50 weight parts of cross-linked povidone, 20 weight parts of sodium croscarmellose, and 5 weight parts of sodium stearyl fumarate.

In a specific embodiment of the present disclosure, the oral solid formulation containing the solid dispersion of total flavonoids of Desmodium Styracifolium may include: 50 weight parts of total flavonoids of Desmodium Styracifolium, 200 weight parts of povidone K₃₀, 100 weight parts of poloxamer 188, 30 weight parts of methoxypolyethylene glycol, 50 weight parts of microcrystalline cellulose, 20 weight parts of sodium croscarmellose, and 5 weight parts of aerosil.

In a specific embodiment of the present disclosure, the oral solid formulation containing the solid dispersion of total flavonoids of Desmodium Styracifolium may include: 66.5 weight parts of total flavonoids of Desmodium Styracifolium, 266 weight parts of povidone K₃₀, 133 weight parts of poloxamer 188, 13.3 weight parts of sodium dodecyl sulfate, 20 weight parts of microcrystalline cellulose, 10 weight parts of sodium carboxymethyl starch, and 8 weight parts of sodium stearyl fumarate.

In a specific embodiment of the present disclosure, the oral solid formulation containing the solid dispersion of total flavonoids of Desmodium Styracifolium may include: 66.5 weight parts of total flavonoids of Desmodium Styracifolium, 266 weight parts of povidone K₃₀, 133 weight parts of poloxamer 188, 39.9 weight parts of sodium dodecyl sulfate, 10 weight parts of lactose, 15 weight parts of sodium croscarmellose, and 6 weight parts of sodium stearyl fumarate.

In a specific embodiment of the present disclosure, the oral solid formulation containing the solid dispersion of total flavonoids of Desmodium Styracifolium may include: 66.5 weight parts of total flavonoids of Desmodium Styracifolium, 266 weight parts of povidone K₃₀, 133 weight parts of poloxamer 188, 66.5 weight parts of sodium dodecyl sulfate, 1 weight part of lactose, 10 weight parts of cross-linked povidone, and 10 weight parts of magnesium stearate.

In a specific embodiment of the present disclosure, the oral solid formulation containing the solid dispersion of total flavonoids of Desmodium Styracifolium may include: 66.5 weight parts of total flavonoids of Desmodium Styracifolium, 266 weight parts of povidone K₃₀, 133 weight parts of poloxamer 188, 39.9 weight parts of Tween 80, 10 weight parts of lactose, 50 weight parts of sodium croscarmellose, and 1 weight part of sodium stearyl fumarate.

In a specific embodiment of the present disclosure, the oral solid formulation containing the solid dispersion of total flavonoids of Desmodium Styracifolium may include: 66.5 weight parts of total flavonoids of Desmodium Styracifolium, 266 weight parts of povidone K₃₀, 133 weight parts of poloxamer 188, 39.9 weight parts of methoxypolyethylene glycol, 15 weight parts of pregelatinized starch, 30 weight parts of sodium croscarmellose, and 5 weight parts of sodium stearyl fumarate.

In a specific embodiment of the present disclosure, the oral solid formulation containing the solid dispersion of total flavonoids of Desmodium Styracifolium may include: 100 weight parts of total flavonoids of Desmodium Styracifolium, 400 weight parts of povidone K₃₀, 200 weight parts of poloxamer 188, 20 weight parts of sodium dodecyl sulfate, 50 weight parts of pregelatinized starch, 20 weight parts of sodium croscarmellose, and 5 weight parts of sodium stearyl fumarate.

In a specific embodiment of the present disclosure, the oral solid formulation containing the solid dispersion of total flavonoids of Desmodium Styracifolium may include: 100 weight parts of total flavonoids of Desmodium Styracifolium, 400 weight parts of povidone K₃₀, 200 weight parts of poloxamer 188, 60 weight parts of sodium dodecyl sulfate, 50 weight parts of lactose, 40 weight parts of cross-linked povidone, and 8 weight parts of sodium stearyl fumarate.

In a specific embodiment of the present disclosure, the oral solid formulation containing the solid dispersion of total flavonoids of Desmodium Styracifolium may include: 100 weight parts of total flavonoids of Desmodium Styracifolium, 400 weight parts of povidone K₃₀, 200 weight parts of poloxamer 188, 100 weight parts of sodium dodecyl sulfate, 10 weight parts of microcrystalline cellulose, 20 weight parts of sodium carboxymethyl starch, and 10 weight parts of sodium stearyl fumarate.

In a specific embodiment of the present disclosure, the oral solid formulation containing the solid dispersion of total flavonoids of Desmodium Styracifolium may include: 100 weight parts of total flavonoids of Desmodium Styracifolium, 400 weight parts of povidone K₃₀, 200 weight parts of poloxamer 188, 60 weight parts of Tween 80, 10 weight parts of lactose, 15 weight parts of sodium croscarmellose, and 8 weight parts of sodium stearyl fumarate.

In a specific embodiment of the present disclosure, the oral solid formulation containing the solid dispersion of total flavonoids of Desmodium Styracifolium may include: 100 weight parts of total flavonoids of Desmodium Styracifolium, 400 weight parts of povidone K₃₀, 200 weight parts of poloxamer 188, 60 weight parts of methoxypolyethylene glycol, 15 weight parts of lactose, 10 weight parts of sodium croscarmellose, and 8 weight parts of sodium stearyl fumarate.

In a specific embodiment of the present disclosure, the oral solid formulation containing the solid dispersion of total flavonoids of Desmodium Styracifolium may include: 133 weight parts of total flavonoids of Desmodium Styracifolium, 532 weight parts of povidone K₃₀, 266 weight parts of poloxamer 188, 26.6 weight parts of sodium dodecyl sulfate, 20 weight parts of lactose, 20 weight parts of sodium croscarmellose, and 8 weight parts of sodium stearyl fumarate.

In a specific embodiment of the present disclosure, the oral solid formulation containing the solid dispersion of total flavonoids of Desmodium Styracifolium may include: 133 weight parts of total flavonoids of Desmodium Styracifolium, 532 weight parts of povidone K₃₀, 266 weight parts of poloxamer 188, 79.8 weight parts of sodium dodecyl sulfate, 20 weight parts of lactose, 25 weight parts of cross-linked povidone, and 9 weight parts of sodium stearyl fumarate.

In a specific embodiment of the present disclosure, the oral solid formulation containing the solid dispersion of total flavonoids of Desmodium Styracifolium may include: 133 weight parts of total flavonoids of Desmodium Styracifolium, 532 weight parts of povidone K₃₀, 266 weight parts of poloxamer 188, 133 weight parts of sodium dodecyl sulfate, 1 weight part of microcrystalline cellulose, 1 weight part of sodium carboxymethyl starch, and 10 weight parts of sodium stearyl fumarate.

In a specific embodiment of the present disclosure, the oral solid formulation containing the solid dispersion of total flavonoids of Desmodium Styracifolium may include: 133 weight parts of total flavonoids of Desmodium Styracifolium, 532 weight parts of povidone K₃₀, 266 weight parts of poloxamer 188, 79.8 weight parts of Tween 80, 10 weight parts of lactose, 10 weight parts of sodium croscarmellose, and 8 weight parts of magnesium stearate.

In a specific embodiment of the present disclosure, the oral solid formulation containing the solid dispersion of total flavonoids of Desmodium Styracifolium may include: 133 weight parts of total flavonoids of Desmodium Styracifolium, 532 weight parts of povidone K₃₀, 266 weight parts of poloxamer 188, 79.8 weight parts of methoxypolyethylene glycol, 5 weight parts of pregelatinized starch, 5 weight parts of cross-linked povidone, and 5 weight parts of magnesium stearate.

In a second aspect of the present disclosure, a method for preparing an oral solid formulation containing total flavonoids of Desmodium Styracifolium is provided, the method includes: providing the total flavonoids of Desmodium Styracifolium in a form of alcohol extract of Desmodium Styracifolium; and formulating the alcohol extract of Desmodium Styracifolium into a capsule or a tablet.

In an embodiment of the present disclosure, the alcohol extract of Desmodium Styracifolium is obtained by the following steps: heating and refluxing a raw material of Desmodium Styracifolium with ethanol having a concentration ranging from 50 to 95% and a weight ranging from 8 to 14 times as heavy as the raw material of Desmodium styracifolium, so as to obtain an extracting solution of Desmodium Styracifolium; concentrating the extracting solution of Desmodium Styracifolium, so as to remove ethanol; and subjecting the extracting solution of Desmodium Styracifolium after concentrated to adsorption onto a macroporous resin column, so as to obtain the alcohol extract of Desmodium Styracifolium.

In an embodiment of the present disclosure, the extracting solution of Desmodium Styracifolium is obtained by: heating and refluxing the raw material of Desmodium Styracifolium for extraction, 1 to 3 times with 1 to 3 hours for each time, with ethanol having the concentration ranging from 50% to 95% and the weight ranging from 8 to 14 times as heavy as the raw material of Desmodium styracifolium, to obtain alcohol extracting solutions of Desmodium styracifolium, and mixing the alcohol extracting solutions.

In an embodiment of the present disclosure, formulating the alcohol extract of Desmodium Styracifolium into the capsule or the table further includes: mixing the alcohol extract of Desmodium Styracifolium with a solid dispersion carrier and a surfactant, and preparing a solid dispersion containing total flavonoids of Desmodium Styracifolium via a solid dispersion technology; mixing the solid dispersion containing total flavonoids of Desmodium Styracifolium with a filler and a disintegrant followed by preparing a soften material, granulating and size stabilizing, and then mixing with a lubricant, so as to obtain a mixture; and capsulizing the mixture with an encapsulating machine so as to obtain a capsule containing total flavonoids of Desmodium Styracifolium, or tableting the mixture with a tableting machine so as to obtain a tablet containing total flavonoids of Desmodium Styracifolium.

In an embodiment of the present disclosure, the solid dispersion carrier is at least one of povidone K₃₀ and poloxamer 188.

In an embodiment of the present disclosure, the surfactant is at least one of Tween 80, methoxypolyethylene glycol, and sodium dodecyl sulfate.

In an embodiment of the present disclosure, the filler is at least one of microcrystalline cellulose, lactose, and pregelatinized starch.

In an embodiment of the present disclosure, the disintegrant is at least one of sodium croscarmellose, cross-linked povidone, and sodium carboxymethyl starch.

In an embodiment of the present disclosure, the lubricant is at least one of magnesium stearate, aerosil, and sodium stearyl fumarate.

The inventor has obtained a tablet containing total flavonoids of Desmodium Styracifolium with a content uniformity meeting requirements for a tablet recorded in Appendix I D, Part 1 of Chinese Pharmacopoeia (2010 edition) by a conventional wet granulation experiment with the following steps: evenly mixing lactose, microcrystalline cellulose with the total flavonoids of Desmodium Styracifolium, then adding a aqueous solution of povidone K₃₀ and stirring to be uniform, followed by preparing a soften material, granulating, stoving, size stabilizing, and tableting. However, the total flavonoid extract of Desmodium Styracifolium is difficult to be dissolved in water and becomes sticky when encountering water (viscosity is increased) in practice, it has been found that, the total flavonoids extract of Desmodium Styracifolium is hardly to be granulated with poor granulating effect during manually granulation process in lab-scale tests. Although the total flavonoids extract of Desmodium Styracifolium can be granulated with improved granulating effects using a granulating machine, it has been detected that a dissolution ration is between 73% and 78% with a relative large difference among batches, thus a resulting product is in danger of becoming unqualified during long term storage.

It has been found through a large number of experiments and studies that, a pharmaceutical formulation with a reliable and stable quality can not be obtained by a conventional preparation method. After shifting the way of thinking, formulas for preparing medicines and formulations containing poorly soluble total flavonoids of Desmodium Styracifolium are further designed and selected with the solid dispersion technology.

An expected beneficial effect has been obtained by the present inventor through a large number of experiments and studies. The present inventor has found through studies and experiments that, the dissolution and absorption of drugs will be promoted with the solid dispersion technology by dispersing the total flavonoid of Desmodium Styracifolium into a composition of hydrophilic solid dispersion carrier materials in a certain ratio, and adding a certain amount of surfactant.

Moreover, the present inventor has surprisingly found that, the dissolution and absorption of total flavonoids of Desmodium Styracifolium can be promoted to a large extent when choosing a composition of povidone K₃₀ and poloxamer 188 (a weight ratio of povidone K₃₀ to poloxamer 188 is 2:1) as the hydrophilic carrier materials of the solid dispersion and adding a surfactant (a weight ratio of the hydrophilic carrier materials of the solid dispersion to the surfactant is 10:1).

A most preferred formula of the solid dispersion containing total flavonoids of Desmodium Styracifolium determined by the present disclosure through experiments and studies is: a weight ratio of total flavonoids of Desmodium Styracifolium, povidone K₃₀, poloxamer 188, and sodium dodecyl sulfate contained the solid dispersion is 1:4:2:0.6, with which the oral solid formulation (capsule or tablet) containing total flavonoids of Desmodium Styracifolium has a high dissolution rate and a good quality stability.

The oral solid formulation (capsule or tablet) containing total flavonoids of Desmodium Styracifolium prepared by the present disclosure with the solid dispersion process has a significantly improved dissolution rate which can be stabled in the range from 88% to 92% and has small differences among different batches, compared to that prepared by an ordinary formulating process.

In an embodiment of the present disclosure, the method for preparing the oral solid formulation (capsule or tablet) containing total flavonoids of Desmodium Styracifolium includes: providing total flavonoids of Desmodium Styracifolium in a form of alcohol extract of Desmodium Styracifolium and a pharmaceutical adjuvant, and formulating the total flavonoids of Desmodium Styracifolium and the pharmaceutical adjuvant into a capsule or a tablet with the solid dispersion technology.

In an embodiment of the present disclosure, a proportion among components in the oral solid formulation containing total flavonoids of Desmodium Styracifolium is: 33 to 133 weight parts of total flavonoids of Desmodium Styracifolium, 198 to 798 weight parts of the solid dispersion carrier, 6.6 to 133 weight parts of the surfactant, 1 to 50 weight parts of filler, 1 to 50 weight parts of disintegrant, and 1 to 10 weight parts of lubricant.

In some embodiments of the present disclosure, preparing the total flavonoids of Desmodium Styracifolium further includes: subjecting the Desmodium Styracifolium to extraction with ethanol, so as to obtain extracting solution of Desmodium Styracifolium, and purifying the extracting solution of Desmodium Styracifolium, so as to obtain extract of Desmodium Styracifolium. In some embodiments of the present disclosure, subjecting the Desmodium Styracifolium to extraction with ethanol further includes: heating and refluxing the raw material of Desmodium Styracifolium for extraction, 1 to 3 times with 1 to 3 hours for each time, with ethanol having the concentration ranging from 50% to 95% and the weight ranging from 8 to 14 times as heavy as the raw material of Desmodium styracifolium, to obtain alcohol extracting solutions of Desmodium styracifolium, and mixing the alcohol extracting solutions. In a specific embodiment of the present disclosure, purifying the extracting solution of Desmodium Styracifolium further includes: concentrating the extracting solution of Desmodium Styracifolium, so as to remove ethanol; and subjecting the extracting solution of Desmodium Styracifolium after concentrated to adsorption onto a macroporous resin column, so as to obtain a purified total flavonoids of Desmodium Styracifolium.

In some specific embodiments, a method for preparing a capsule containing total flavonoids of Desmodium Styracifolium further includes the following steps:

preparing the solid dispersion containing total flavonoids of Desmodium Styracifolium: dissolving the total flavonoids of Desmodium Styracifolium, a solid dispersion carrier and a surfactant in respective formula dosage, with ethanol having a concentration of 50% under stirring, followed by removing the solvent via evaporation under reduced pressure, vacuum drying, smashing and sieving at 40 to 200 meshes, so as to obtain the solid dispersion containing total flavonoids of Desmodium Styracifolium.

preparing granules containing total flavonoids of Desmodium Styracifolium: mixing a filler and a disintegrant with the solid dispersion containing the total flavonoids of Desmodium Styracifolium to be uniform, followed by preparing a soften material, granulating at 10 to 30 meshes, size stabilizing, and mixing with a lubricant to be uniform, so as to obtain the granules containing total flavonoids of Desmodium Styracifolium;

capsulizing: capsulizing the granules containing total flavonoids of Desmodium Styracifolium with an encapsulating machine so as to obtain a capsule containing total flavonoids of Desmodium Styracifolium.

In some embodiments of the present disclosure, a method for preparing a tablet containing total flavonoids of Desmodium Styracifolium further includes the following steps:

preparing the solid dispersion containing total flavonoids of Desmodium Styracifolium: dissolving the total flavonoids of Desmodium Styracifolium, a solid dispersion carrier and a surfactant in respective formula dosage, with ethanol having a concentration of 50% under stirring, followed by removing the solvent via evaporation under reduced pressure, vacuum drying, smashing and sieving at 40 to 200 meshes, so as to obtain the solid dispersion containing total flavonoids of Desmodium Styracifolium.

preparing granules containing total flavonoids of Desmodium Styracifolium: mixing a filler and a disintegrant with the solid dispersion containing the total flavonoids of Desmodium Styracifolium to be uniform, followed by preparing a soften material, granulating at 10 to 30 meshes, size stabilizing, and mixing with a lubricant to be uniform, so as to obtain the granules containing total flavonoids of Desmodium Styracifolium;

tableting: tableting the granules containing total flavonoids of Desmodium Styracifolium with a tableting machine to obtain a tablet containing total flavonoids of Desmodium Styracifolium.

In some embodiments of the present disclosure, in a method for preparing the oral solid formulation containing total flavonoids of Desmodium Styracifolium, the solid dispersion carrier alternatively is at least one of povidone K₃₀, polyethylene glycol 6000, D-mannitol, and poloxamer 188, and alternatively, is a combination of povidone K₃₀ and poloxamer 188.

In some embodiments of the present disclosure, in a method for preparing the oral solid formulation containing total flavonoids of Desmodium Styracifolium, the surfactant alternatively is at least one of Tween 80, methoxypolyethylene glycol, and sodium dodecyl sulfate, and alternatively, is sodium dodecyl sulfate.

In some embodiments of the present disclosure, in a method for preparing the oral solid formulation containing total flavonoids of Desmodium Styracifolium, the filler alternatively is at least one of microcrystalline cellulose, lactose, and pregelatinized starch, and alternatively, is lactose.

In some embodiments of the present disclosure, in a method for preparing the oral solid formulation containing total flavonoids of Desmodium Styracifolium, the disintegrant alternatively is at least one of sodium croscarmellose, cross-linked povidone, and sodium carboxymethyl starch, and alternatively, is sodium croscarmellose.

In some embodiments of the present disclosure, in a method for preparing the oral solid formulation containing total flavonoids of Desmodium Styracifolium, the lubricant alternatively is at least one of magnesium stearate, aerosil, and sodium stearyl fumarate, and alternatively, is sodium stearyl fumarate.

In some embodiments of the present disclosure, in a method for preparing the oral solid formulation containing total flavonoids of Desmodium Styracifolium, a weight ratio of total flavonoids of Desmodium Styracifolium as raw material to a hydrophilic carrier as the solid dispersion is in a range from 1:5 to 12.

In some embodiments of the present disclosure, in a method for preparing the oral solid formulation containing total flavonoids of Desmodium Styracifolium, alternatively, the weight ratio of total flavonoids of Desmodium Styracifolium to the hydrophilic carrier as the solid dispersion is in a range from 1:5.5 to 8.

In some embodiments of the present disclosure, in a method for preparing the oral solid formulation containing total flavonoids of Desmodium Styracifolium, alternatively, the weight ratio of total flavonoids of Desmodium Styracifolium to the hydrophilic carrier as the solid dispersion is 1:6.

In an embodiment of the present disclosure, alternatively, the composition of povidone K₃₀ and poloxamer 188 is used as a hydrophilic carrier material of the solid dispersion, into which the total flavonoids of Desmodium Styracifolium can be ensured to be dispersed with a high dispersibility, in addition, by further adding sodium dodecyl sulfate as surfactant which has a solubilizing effect itself in suitable amount, poorly soluble drugs may achieve a significantly increased solubility, a more effectively reduced surface contact angle, an improved wettability, thus an increased solubility of total flavonoids of Desmodium Styracifolium, and effectively promoted dispersion and absorption.

In some embodiments of the present disclosure, a weight ratio of povidone K₃₀ to poloxamer 188 is in a range from 1:0.05 to 20.

In some embodiments of the present disclosure, alternatively, the weight ratio of povidone K₃₀ to poloxamer 188 is in a range from 1:0.2 to 4.

In some embodiments of the present disclosure, alternatively, the weight ratio of povidone K₃₀ to poloxamer 188 is 1:0.5.

In some embodiments of the present disclosure, in a method for preparing the oral solid formulation containing total flavonoids of Desmodium Styracifolium, a weight ratio of total flavonoids of Desmodium Styracifolium to the surfactant is in a range from 1:0.05 to 1.

In some embodiments of the present disclosure, in a method for preparing the oral solid formulation containing total flavonoids of Desmodium Styracifolium, alternatively, the weight ratio of total flavonoids of Desmodium Styracifolium to the surfactant is in a range from 1:0.2 to 0.8.

In some embodiments of the present disclosure, in a method for preparing the oral solid formulation containing total flavonoids of Desmodium Styracifolium, alternatively, the weight ratio of total flavonoids of Desmodium Styracifolium to the surfactant is 1:0.6. At this time, a weight ratio of the hydrophilic carrier as the solid dispersion to the surfactant is 10:1.

In some embodiment of the present disclosure, a proportion among components in the oral solid formulation containing total flavonoids of Desmodium Styracifolium is: 33 to 133 weight parts of total flavonoids of Desmodium Styracifolium, 198 to 798 weight parts of the solid dispersion carrier, 6.6 to 133 weight parts of the surfactant, 1 to 50 weight parts of filler, 1 to 50 weight parts of disintegrant, and 1 to 10 weight parts of lubricant.

In a specific embodiment of the present disclosure, a proportion among components in the oral solid formulation containing total flavonoids of Desmodium Styracifolium is: 33 weight parts of total flavonoids of Desmodium Styracifolium, 132 weight parts of povidone K₃₀, 66 weight parts of poloxamer 188, 6.6 weight parts of sodium dodecyl sulfate, 50 weight parts of lactose, 20 weight parts of cross-linked povidone, and 1 weight part of aerosil.

In a specific embodiment of the present disclosure, a proportion among components in the oral solid formulation containing total flavonoids of Desmodium Styracifolium is: 33 weight parts of total flavonoids of Desmodium Styracifolium, 132 weight parts of povidone K₃₀, 66 weight parts of poloxamer 188, 19.8 weight parts of sodium dodecyl sulfate, 30 weight parts of lactose, 20 weight parts of sodium croscarmellose, and 3 weight parts of sodium stearyl fumarate.

In a specific embodiment of the present disclosure, a proportion among components in the oral solid formulation containing total flavonoids of Desmodium Styracifolium is: 33 weight parts of total flavonoids of Desmodium Styracifolium, 132 weight parts of povidone K₃₀, 66 weight parts of poloxamer 188, 33 weight parts of sodium dodecyl sulfate, 10 weight parts of microcrystalline cellulose, 20 weight parts of sodium croscarmellose, and 2 weight parts of magnesium stearate.

In a specific embodiment of the present disclosure, a proportion among components in the oral solid formulation containing total flavonoids of Desmodium Styracifolium is: 33 weight parts of total flavonoids of Desmodium Styracifolium, 132 weight parts of povidone K₃₀, 66 weight parts of poloxamer 188, 19.8 weight parts of Tween 80, 40 weight parts of lactose, 5 weight parts of sodium carboxymethyl starch, and 4 weight parts of sodium stearyl fumarate.

In a specific embodiment of the present disclosure, a proportion among components in the oral solid formulation containing total flavonoids of Desmodium Styracifolium is: 33 weight parts of total flavonoids of Desmodium Styracifolium, 132 weight parts of povidone K₃₀, 66 weight parts of poloxamer 188, 19.8 weight parts of methoxypolyethylene glycol, 20 weight parts of microcrystalline cellulose, 15 weight parts of sodium carboxymethyl starch, and 3 weight parts of sodium stearyl fumarate.

In a specific embodiment of the present disclosure, a proportion among components in the oral solid formulation containing total flavonoids of Desmodium Styracifolium is: 50 weight parts of total flavonoids of Desmodium Styracifolium, 200 weight parts of povidone K₃₀, 100 weight parts of poloxamer 188, 10 weight parts of sodium dodecyl sulfate, 50 weight parts of lactose, 20 weight parts of sodium croscarmellose, and 5 weight parts of sodium stearyl fumarate.

In a specific embodiment of the present disclosure, a proportion among components in the oral solid formulation containing total flavonoids of Desmodium Styracifolium is: 50 weight parts of total flavonoids of Desmodium Styracifolium, 200 weight parts of povidone K₃₀, 100 weight parts of poloxamer 188, 30 weight parts of sodium dodecyl sulfate, 50 weight parts of lactose, 20 weight parts of sodium croscarmellose, and 5 weight parts of sodium stearyl fumarate.

In a specific embodiment of the present disclosure, a proportion among components in the oral solid formulation containing total flavonoids of Desmodium Styracifolium is: 50 weight parts of total flavonoids of Desmodium Styracifolium, 200 weight parts of povidone K₃₀, 100 weight parts of poloxamer 188, 50 weight parts of sodium dodecyl sulfate, 50 weight parts of lactose, 20 weight parts of sodium croscarmellose, and 5 weight parts of sodium stearyl fumarate.

In a specific embodiment of the present disclosure, a proportion among components in the oral solid formulation containing total flavonoids of Desmodium Styracifolium is: 50 weight parts of total flavonoids of Desmodium Styracifolium, 200 weight parts of povidone K₃₀, 100 weight parts of poloxamer 188, 30 weight parts of Tween 80, 50 weight parts of lactose, 20 weight parts of sodium croscarmellose, and 5 weight parts of sodium stearyl fumarate.

In a specific embodiment of the present disclosure, a proportion among components in the oral solid formulation containing total flavonoids of Desmodium Styracifolium is: 50 weight parts of total flavonoids of Desmodium Styracifolium, 200 weight parts of povidone K₃₀, 100 weight parts of poloxamer 188, 30 weight parts of methoxypolyethylene glycol, 50 weight parts of lactose, 20 weight parts of sodium croscarmellose, and 5 weight parts of sodium stearyl fumarate.

In a specific embodiment of the present disclosure, a proportion among components in the oral solid formulation containing total flavonoids of Desmodium Styracifolium is: 50 weight parts of total flavonoids of Desmodium Styracifolium, 200 weight parts of povidone K₃₀, 100 weight parts of poloxamer 188, 30 weight parts of sodium dodecyl sulfate, 50 weight parts of pregelatinized starch, 20 weight parts of sodium croscarmellose, and 5 weight parts of sodium stearyl fumarate.

In a specific embodiment of the present disclosure, a proportion among components in the oral solid formulation containing total flavonoids of Desmodium Styracifolium is: 50 weight parts of total flavonoids of Desmodium Styracifolium, 200 weight parts of povidone K₃₀, 100 weight parts of poloxamer 188, 50 weight parts of sodium dodecyl sulfate, 50 weight parts of cross-linked povidone, 20 weight parts of sodium croscarmellose, and 5 weight parts of sodium stearyl fumarate.

In a specific embodiment of the present disclosure, a proportion among components in the oral solid formulation containing total flavonoids of Desmodium Styracifolium is: 50 weight parts of total flavonoids of Desmodium Styracifolium, 200 weight parts of povidone K₃₀, 100 weight parts of poloxamer 188, 30 weight parts of methoxypolyethylene glycol, 50 weight parts of microcrystalline cellulose, 20 weight parts of sodium croscarmellose, and 5 weight parts of aerosil.

In a specific embodiment of the present disclosure, a proportion among components in the oral solid formulation containing total flavonoids of Desmodium Styracifolium is: 66.5 weight parts of total flavonoids of Desmodium Styracifolium, 266 weight parts of povidone K₃₀, 133 weight parts of poloxamer 188, 13.3 weight parts of sodium dodecyl sulfate, 20 weight parts of microcrystalline cellulose, 10 weight parts of sodium carboxymethyl starch, and 8 weight parts of sodium stearyl fumarate.

In a specific embodiment of the present disclosure, a proportion among components in the oral solid formulation containing total flavonoids of Desmodium Styracifolium is: 66.5 weight parts of total flavonoids of Desmodium Styracifolium, 266 weight parts of povidone K₃₀, 133 weight parts of poloxamer 188, 39.9 weight parts of sodium dodecyl sulfate, 10 weight parts of lactose, 15 weight parts of sodium croscarmellose, and 6 weight parts of sodium stearyl fumarate.

In a specific embodiment of the present disclosure, a proportion among components in the oral solid formulation containing total flavonoids of Desmodium Styracifolium is: 66.5 weight parts of total flavonoids of Desmodium Styracifolium, 266 weight parts of povidone K₃₀, 133 weight parts of poloxamer 188, 66.5 weight parts of sodium dodecyl sulfate, 1 weight part of lactose, 10 weight parts of cross-linked povidone, and 10 weight parts of magnesium stearate.

In a specific embodiment of the present disclosure, a proportion among components in the oral solid formulation containing total flavonoids of Desmodium Styracifolium is: 66.5 weight parts of total flavonoids of Desmodium Styracifolium, 266 weight parts of povidone K₃₀, 133 weight parts of poloxamer 188, 39.9 weight parts of Tween 80, 10 weight parts of lactose, 50 weight parts of sodium croscarmellose, and 1 weight part of sodium stearyl fumarate.

In a specific embodiment of the present disclosure, a proportion among components in the oral solid formulation containing total flavonoids of Desmodium Styracifolium is: 66.5 weight parts of total flavonoids of Desmodium Styracifolium, 266 weight parts of povidone K₃₀, 133 weight parts of poloxamer 188, 39.9 weight parts of methoxypolyethylene glycol, 15 weight parts of pregelatinized starch, 30 weight parts of sodium croscarmellose, and 5 weight parts of sodium stearyl fumarate.

In a specific embodiment of the present disclosure, a proportion among components in the oral solid formulation containing total flavonoids of Desmodium Styracifolium is: 100 weight parts of total flavonoids of Desmodium Styracifolium, 400 weight parts of povidone K₃₀, 200 weight parts of poloxamer 188, 20 weight parts of sodium dodecyl sulfate, 50 weight parts of pregelatinized starch, 20 weight parts of sodium croscarmellose, and 5 weight parts of sodium stearyl fumarate.

In a specific embodiment of the present disclosure, a proportion among components in the oral solid formulation containing total flavonoids of Desmodium Styracifolium is: 100 weight parts of total flavonoids of Desmodium Styracifolium, 400 weight parts of povidone K₃₀, 200 weight parts of poloxamer 188, 60 weight parts of sodium dodecyl sulfate, 50 weight parts of lactose, 40 weight parts of cross-linked povidone, and 8 weight parts of sodium stearyl fumarate.

In a specific embodiment of the present disclosure, a proportion among components in the oral solid formulation containing total flavonoids of Desmodium Styracifolium is: 100 weight parts of total flavonoids of Desmodium Styracifolium, 400 weight parts of povidone K₃₀, 200 weight parts of poloxamer 188, 100 weight parts of sodium dodecyl sulfate, 10 weight parts of microcrystalline cellulose, 20 weight parts of sodium carboxymethyl starch, and 10 weight parts of sodium stearyl fumarate.

In a specific embodiment of the present disclosure, a proportion among components in the oral solid formulation containing total flavonoids of Desmodium Styracifolium is: 100 weight parts of total flavonoids of Desmodium Styracifolium, 400 weight parts of povidone K₃₀, 200 weight parts of poloxamer 188, 60 weight parts of Tween 80, 10 weight parts of lactose, 15 weight parts of sodium croscarmellose, and 8 weight parts of sodium stearyl fumarate.

In a specific embodiment of the present disclosure, a proportion among components in the oral solid formulation containing total flavonoids of Desmodium Styracifolium is: 100 weight parts of total flavonoids of Desmodium Styracifolium, 400 weight parts of povidone K₃₀, 200 weight parts of poloxamer 188, 60 weight parts of methoxypolyethylene glycol, 15 weight parts of lactose, 10 weight parts of sodium croscarmellose, and 8 weight parts of sodium stearyl fumarate. In a specific embodiment of the present disclosure, a proportion among components in the oral solid formulation containing total flavonoids of Desmodium Styracifolium is: 133 weight parts of total flavonoids of Desmodium Styracifolium, 532 weight parts of povidone K₃₀, 266 weight parts of poloxamer 188, 26.6 weight parts of sodium dodecyl sulfate, 20 weight parts of lactose, 20 weight parts of sodium croscarmellose, and 8 weight parts of sodium stearyl fumarate.

In a specific embodiment of the present disclosure, a proportion among components in the oral solid formulation containing total flavonoids of Desmodium Styracifolium is: 133 weight parts of total flavonoids of Desmodium Styracifolium, 532 weight parts of povidone K₃₀, 266 weight parts of poloxamer 188, 79.8 weight parts of sodium dodecyl sulfate, 20 weight parts of lactose, 25 weight parts of cross-linked povidone, and 9 weight parts of sodium stearyl fumarate.

In a specific embodiment of the present disclosure, a proportion among components in the oral solid formulation containing total flavonoids of Desmodium Styracifolium is: 133 weight parts of total flavonoids of Desmodium Styracifolium, 532 weight parts of povidone K₃₀, 266 weight parts of poloxamer 188, 133 weight parts of sodium dodecyl sulfate, 1 weight part of microcrystalline cellulose, 1 weight part of sodium carboxymethyl starch, and 10 weight parts of sodium stearyl fumarate.

In a specific embodiment of the present disclosure, a proportion among components in the oral solid formulation containing total flavonoids of Desmodium Styracifolium is: 133 weight parts of total flavonoids of Desmodium Styracifolium, 532 weight parts of povidone K₃₀, 266 weight parts of poloxamer 188, 79.8 weight parts of Tween 80, 10 weight parts of lactose, 10 weight parts of sodium croscarmellose, and 8 weight parts of magnesium stearate.

In a specific embodiment of the present disclosure, a proportion among components in the oral solid formulation containing total flavonoids of Desmodium Styracifolium is: 133 weight parts of total flavonoids of Desmodium Styracifolium, 532 weight parts of povidone K₃₀, 266 weight parts of poloxamer 188, 79.8 weight parts of methoxypolyethylene glycol, 5 weight parts of pregelatinized starch, 5 weight parts of cross-linked povidone, and 5 weight parts of magnesium stearate.

In specific, in some embodiments of the present disclosure, a method for preparing a capsule containing total flavonoids of Desmodium Styracifolium may include the following steps:

a. preparing total flavonoids of Desmodium Styracifolium: weighing a raw material of Desmodium Styracifolium, heating and refluxing the raw material for extraction, 1 to 3 times with 1 to 3 hours for each time, at a temperature of 50° C. to 60° C. with ethanol having a concentration ranging from 50% to 95% and a weight ranging from 8 to 14 times as heavy as the raw material, so as to obtain alcohol extracting solutions of Desmodium Styracifolium followed by mixing; concentrating the alcohol extracting solution to be of a volume 2 to 8 times the weight of the raw material followed by still standing and filtering to obtain a filtrate; subjecting the filtrate to adsorption onto an AB-8 macroporous resin column at a flow rate ranging from 1 to 3 column bed volumes per hour, eluting and purifying with water having a volume ranging from 8 to 12 times the weight of filled resin, and eluting with ethanol having a concentration of 40% to 95% and a volume ranging from 6 to 10 column bed volumes at a flow rate ranging from 2 to 4 column bed volumes per hour, to obtain an eluted solution; and concentrating the eluted solution to recycle ethanol and to obtain a concentrated solution with a relative density ranging from 1.10 to 1.30 followed by drying and then smashing, so as to obtain total flavonoids of Desmodium Styracifolium,

b. preparing a solid dispersion containing total flavonoids of Desmodium Styracifolium: dissolving the total flavonoids of Desmodium Styracifolium, a solid dispersion carrier and a surfactant in respective formula dosage, sieved at 40 to 100 meshes in advance respectively, with ethanol having a concentration of 50% under stirring and at a temperature of 50° C. to 75° C., followed by removing the solvent via evaporation under reduced pressure at a temperature of 30° C. to 75° C., vacuum drying at a temperature of 30° C. to 60° C., smashing and sieving at 40 to 200 meshes, so as to obtain the solid dispersion containing total flavonoids of Desmodium Styracifolium;

c. preparing granules containing total flavonoids of Desmodium Styracifolium: mixing a filler and a disintegrant, sieved at 40 to 100 meshes in advance, respectively, with the solid dispersion containing total flavonoids of Desmodium Styracifolium to be uniform, followed by preparing a soften material, granulating at 10 to 30 meshes, drying at a temperature of 30° C. to 75° C., size stabilizing, and mixing with a lubricant to be uniform, so as to obtain the granules containing total flavonoids of Desmodium Styracifolium;

d. capsulizing: capsulizing the granules containing total flavonoids of Desmodium Styracifolium with an encapsulating machine so as to obtain a capsule containing total flavonoids of Desmodium Styracifolium.

In specific, in some embodiments of the present disclosure, a method for preparing a capsule containing total flavonoids of Desmodium Styracifolium may include the following steps:

a. preparing the total flavonoids of Desmodium Styracifolium: weighing a raw material of Desmodium Styracifolium in a formula dosage, heating and refluxing at a temperature of 55° C. for 2 hours for first extraction with ethanol having a concentration of 80% and a weight 12 times as heavy as the raw material, heating and refluxing at a temperature of 55° C. for 1.5 hours for second extraction with ethanol having a concentration of 80% and a weight 10 times as heavy as the raw material, so as to obtain alcohol extracting solutions of Desmodium Styracifolium followed by mixing; concentrating the alcohol extracting solution to be of a volume 5 times the weight of the raw material followed by still standing and filtering, to obtain a filtrate; subjecting the filtrate to adsorption onto an AB-8 macroporous resin column at a flow rate of 3 column bed volumes per hour, eluting and purifying with water having a volume 10 times the weight of filled resin, and eluting with ethanol having a concentration of 60% and a volume of 8 column bed volumes at a flow rate of 3 column bed volumes per hour, to obtain an eluted solution; and concentrating the eluted solution to recycle ethanol and to obtain a concentrated solution with a relative density of 1.22, followed by drying under reduced pressure at a temperature of 75° C. and then smashing to obtain total flavonoids of Desmodium Styracifolium;

b. preparing a solid dispersion containing total flavonoids of Desmodium Styracifolium: dissolving the total flavonoids of Desmodium Styracifolium, povidone K₃₀, poloxamer 188 and sodium dodecyl sulfate in respective formula dosage, sieved at 80 meshes in advance respectively, with ethanol having a concentration of 50% under stirring and at a temperature of 65° C., followed by removing the solvent via evaporation under reduced pressure at a temperature of 50° C., vacuum drying at a temperature of 40° C., smashing and sieving at 80 meshes, so as to obtain the solid dispersion containing total flavonoids of Desmodium Styracifolium;

c. preparing granules containing total flavonoids of Desmodium Styracifolium: mixing lactose and sodium croscarmellose, sieved at 80 meshes in advance respectively, with the solid dispersion containing the total flavonoids of Desmodium Styracifolium to be uniform, followed by preparing a soften material, granulating at 20 meshes, drying at a temperature of 55° C., size stabilizing, and mixing with aerosil to be uniform, so as to obtain the granules containing total flavonoids of Desmodium Styracifolium;

d. capsulizing: capsulizing the granules containing total flavonoids of Desmodium Styracifolium with an encapsulating machine so as to obtain a capsule containing total flavonoids of Desmodium Styracifolium.

In specific, in some embodiments of the present disclosure, a method for preparing a tablet containing total flavonoids of Desmodium Styracifolium may include the following steps:

a. preparing total flavonoids of Desmodium Styracifolium: weighing a raw material of Desmodium Styracifolium, heating and refluxing the raw material for extraction, 1 to 3 times with 1 to 3 hours for each time, at a temperature of 50° C. to 60° C. with ethanol having a concentration ranging from 50% to 95% and a weight ranging from 8 to 14 times as heavy as the raw material, so as to obtain alcohol extracting solutions of Desmodium Styracifolium followed by mixing; concentrating the alcohol extracting solution to be of a volume 2 to 8 times the weight of the raw material followed by still standing and filtering to obtain a filtrate; subjecting the filtrate to adsorption onto an AB-8 macroporous resin column at a flow rate ranging from 1 to 3 column bed volumes per hour, eluting and purifying with water having a volume ranging from 8 to 12 times the weight of filled resin, and eluting with ethanol having a concentration of 40% to 95% and a volume ranging from 6 to 10 column bed volumes at a flow rate ranging from 2 to 4 column bed volumes per hour, to obtain an eluted solution; and concentrating the eluted solution to recycle ethanol and to obtain a concentrated solution with a relative density ranging from 1.10 to 1.30 followed by drying and then smashing, so as to obtain total flavonoids of Desmodium Styracifolium,

b. preparing a solid dispersion containing total flavonoids of Desmodium Styracifolium: dissolving the total flavonoids of Desmodium Styracifolium, a solid dispersion carrier and a surfactant in respective formula dosage, sieved at 40 to 100 meshes in advance respectively, with ethanol having a concentration of 50% under stirring and at a temperature of 50° C. to 75° C., followed by removing the solvent via evaporation under reduced pressure at a temperature of 30° C. to 75° C., vacuum drying at a temperature of 30° C. to 60° C., smashing and sieving at 40 to 200 meshes, so as to obtain the solid dispersion containing total flavonoids of Desmodium Styracifolium;

c. preparing granules containing total flavonoids of Desmodium Styracifolium: mixing a filler and a disintegrant, sieved at 40 to 100 meshes in advance, respectively, with the solid dispersion containing total flavonoids of Desmodium Styracifolium to be uniform, followed by preparing a soften material, granulating at 10 to 30 meshes, drying at a temperature of 30° C. to 75° C., size stabilizing, and mixing with a lubricant to be uniform, so as to obtain the granules containing total flavonoids of Desmodium Styracifolium;

d. tableting: tableting the granules containing total flavonoids of Desmodium Styracifolium with a tableting machine to obtain a tablet containing total flavonoids of Desmodium Styracifolium.

In specific, in some embodiments of the present disclosure, a method for preparing a tablet containing total flavonoids of Desmodium Styracifolium may include the following steps:

a. preparing the total flavonoids of Desmodium Styracifolium: weighing a raw material of Desmodium Styracifolium in a formula dosage, heating and refluxing at a temperature of 55° C. for 2 hours for first extraction with ethanol having a concentration of 80% and a weight 12 times as heavy as the raw material, heating and refluxing at a temperature of 55° C. for 1.5 hours for second extraction with ethanol having a concentration of 80% and a weight 10 times as heavy as the raw material, so as to obtain alcohol extracting solutions of Desmodium Styracifolium followed by mixing; concentrating the alcohol extracting solution to be of a volume 5 times the weight of the raw material followed by still standing and filtering, to obtain a filtrate; subjecting the filtrate to adsorption onto an AB-8 macroporous resin column at a flow rate of 3 column bed volumes per hour, eluting and purifying with water having a volume 10 times the weight of filled resin, and eluting with ethanol having a concentration of 60% and a volume of 8 column bed volumes at a flow rate of 3 column bed volumes per hour, to obtain an eluted solution; and concentrating the eluted solution to recycle ethanol and to obtain a concentrated solution with a relative density of 1.22, followed by drying under reduced pressure at a temperature of 75° C. and then smashing to obtain total flavonoids of Desmodium Styracifolium;

b. preparing a solid dispersion containing total flavonoids of Desmodium Styracifolium: dissolving the total flavonoids of Desmodium Styracifolium, povidone K₃₀, poloxamer 188 and sodium dodecyl sulfate in respective formula dosage, sieved at 80 meshes in advance respectively, with ethanol having a concentration of 50% under stirring and at a temperature of 65° C., followed by removing the solvent via evaporation under reduced pressure at a temperature of 50° C., vacuum drying at a temperature of 40° C., smashing and sieving at 80 meshes, so as to obtain the solid dispersion containing total flavonoids of Desmodium Styracifolium;

c. preparing granules containing total flavonoids of Desmodium Styracifolium: mixing lactose and sodium croscarmellose, sieved at 80 meshes in advance respectively, with the solid dispersion containing the total flavonoids of Desmodium Styracifolium to be uniform, followed by preparing a soften material, granulating at 20 meshes, drying at a temperature of 55° C., size stabilizing, and mixing with aerosil to be uniform, so as to obtain the granules containing total flavonoids of Desmodium Styracifolium;

d. tableting: tableting the granules containing total flavonoids of Desmodium Styracifolium with a tableting machine to obtain the tablet containing total flavonoids of Desmodium Styracifolium.

Three batches of capsules containing total flavonoids of Desmodium Styracifolium are prepared by the present inventor with the method according to embodiments of the present disclosure, which are subjected to a preliminary investigation on stability. According to the requirement of “Guiding Principle of Pharmaceutical stability Test” (Appendix XIXC, part 2 of Chinese Pharmacopoeia (2010 edition)), influencing factor test, acceleration test and long term test are performed, respectively, and the results show that the capsules containing total flavonoids of Desmodium Styracifolium are stable under lighting condition. A variety of physical chemistry indexes of the capsule have no significant change at a high temperature of 60° C., a relative humidity of 75% for 10 days, under acceleration condition of a temperature of 40° C. for 6 months, or under a long term condition for 6 months.

Three batches of tablets containing total flavonoids of Desmodium Styracifolium are prepared by the present inventor with the method according to embodiments of the present disclosure, which are subjected to a preliminary investigation on stability. According to the requirement of “Guiding Principle of Pharmaceutical stability Test” (Appendix XIXC, part 2 of Chinese Pharmacopoeia (2010 edition)), influencing factor test, acceleration test and long term test are performed, respectively, and the results show that the tablets containing total flavonoids of Desmodium Styracifolium are stable under lighting condition. A variety of physical chemistry indexes of the tablets have no significant change at a high temperature of 60° C., a relative humidity of 75% for 10 days, under acceleration condition of a temperature of 40° C. for 6 months, or under a long term condition for 6 months.

Results obtained from formula screening, quality and stability studies have indicated that, the oral solid formulation containing total flavonoids of Desmodium Styracifolium of the present disclosure has a plurality of advantages, such as reasonable formula, feasible process, reduced production costs, controllable quality tested by product quality standards and stable quality under proposed conditions, better dissolution rate, bioavailability, absorption velocity, industrialization than the prior art, moreover, the solid dispersion containing total flavonoids of Desmodium Styracifolium of the present disclosure has obtained an unexpected technical effect when its formula is: total flavonoids of Desmodium Styracifolium: povidone K₃₀: poloxamer 188: sodium dodecyl sulfate=1:4:2:0.6 (by weight).

In a third aspect of the present disclosure, use of total flavonoids of Desmodium Styracifolium in medicament is provided. According to embodiments of the present disclosure, The oral solid formulation containing total flavonoids of Desmodium Styracifolium prepared in the present disclosure may be used in preparation of clinical therapeutic medicament for scavenging dampness and heat or expelling stone through diuresis (stagnation of dampness-heat).

Based on general pharmacological experiments performed according to embodiments of the present disclosure, after administration of the total flavonoids of Desmodium Styracifolium, there is no obvious change in behaviour, reaction, action, emotion and gait of the animal, and there is no effect on spontaneous activity of the animal, on excitability to central nervous system of the animal or on gastrointestinal movement of the mouse. The results from pharmacological experiments according to embodiments of the present disclosure show that: the total flavonoids of Desmodium Styracifolium may obviously inhibit an amount of calcium oxalate crystalline polymer in kidney, and decrease formation rate of kidney stone and reduce content of creatinine and uric acid, thus improving the kidney function of rat. The total flavonoids of Desmodium Styracifolium may have functions in dissolving stones and reducing formation of new stones, and may also have diuretic effect. Moreover, the total flavonoids of Desmodium Styracifolium may reduce welling degree and swelling rate caused by injecting fresh egg albumen to toes of rats, which indicates that the total flavonoids of Desmodium Styracifolium may have certain anti-inflammatory effect and have obvious inhibiting effect on proliferation of granulation tissue.

According to embodiments of the present disclosure, acute toxicity tests were performed to animals for evaluating safety of the total flavonoids of Desmodium Styracifolium. Mice were administrated with total flavonoids of Desmodium Styracifolium by gavage for acute toxicity observation, corresponding results show that the total flavonoids of Desmodium Styracifolium are substantially nontoxic to the mice. Rats were administrated with total flavonoids of Desmodium Styracifolium by gavage for acute toxicity observation, corresponding results show that there is no server acute toxicity for the rats administrated with total flavonoids of Desmodium Styracifolium. In long term toxicity tests, the total flavonoids of Desmodium Styracifolium have also been proven to be safe for animals.

Similarly, the granula containing solid dispersion of total flavonoids of Desmodium Styracifolium obtained by using the solid dispersion technology and formulating process for formulating the oral solid formulation containing total flavonoids of Desmodium Styracifolium of the present disclosure is of a good dissolution rate, an absorption velocity, a stable quality, and is easy to industrialization.

According to embodiments of the present disclosure, there is provided the preparing process for extracting and separating insoluble total flavonoids of Desmodium Styracifolium (by means of screening the total flavonoids of Desmodium Styracifolium as active ingredient from the raw material and macroporous resin technique) and a granulating process for preparing Chinese medicament with the fluidized bed, such that the capsule containing total flavonoids of Desmodium Styracifolium is developed as a modern and new Chinese medicament capable of effectively treating urinary stones with high dissolution rate and quality stability

The present disclosure has studied a preparing process for extracting and separating poor soluble total flavonoids of Desmodium Styracifolium by screening the total flavonoids of Desmodium Styracifolium as active ingredient from the raw material and macroporous resin technique, and using solid dispersion technology, further developed a tablet containing the total flavonoids of Desmodium Styracifolium, a new modern Chinese medicine drug, with a remarkable curative effect on anti-urolithiasis, a good dissolution and stable quality, and is perfectly suitable to industrial production process.

The oral solid formulation containing total flavonoids of Desmodium Styracifolium prepared by the present disclosure with solid dispersion technology has the following advantages: high dissolution rate, significant clinical effect, less adverse reactions, better therapeutic effect on urinary stone disease (particularly renal pelvis and ureteral calculi disease) than stranguria-treating and calculus-removing tablet. Moreover, The oral solid formulation containing total flavonoids of Desmodium Styracifolium of the present disclosure has a clear active ingredient and content thereof, a stable and controllable quality; and is safe, effective, cheap, economical, practical, and is easy to administrate and simple to use.

As compared with the related art, the technical solution of the present disclosure has advantages as described below:

1. According to embodiments of the present disclosure, in the process for extracting and purifying the raw material, ethanol is used as an extraction solvent for extracting the raw material of Desmodium Styracifolium, and the extracting solution is purified by a macroporous adsorption resin to obtain the active ingredient of Desmodium Styracifolium, i.e., the total flavonoids of Desmodium Styracifolium. As compared with a water-extraction and alcohol-precipitation method for extracting, the active ingredient basis of the extract by such a process is clear and the quality standard is controllable, thus decreasing the dosage for clinical administration and reducing clinical side effects.

2. As compared with a process of ethanol extracting and macroporous resin purifying in the related art, ethanol is recycled from the extracting solution in embodiments of the present disclosure, so that the extracting solution is concentrated to be of a certain volume (5 times of the weight of the raw material) as a consequence, which can be directed purified by the macroporous resin without special concentrating and drying for into extractum, thus saving time for preparation. Besides, after purified by the macroporous resin, the active ingredient with a high content is eluted even using ethanol with the same concentration, which is a simple process and has a good operability as compared with gradient dilution using ethanol with different concentrations. Thirdly, the total flavonoids of Desmodium Styracifolium (i.e., the active ingredient of Desmodium Styracifolium) is obtained by recycling ethanol from the eluted solution and directly drying under reduced pressure without any solvent for processing, thus saving consumption in the preparation. In the view of scale production, the above mentioned process for extracting and purifying decreases production costs, shortens production period, which is simple, convenient and practical, thus meeting requirements to modern industry of Chinese medicine.

3. According to embodiments of the present disclosure, the method includes extracting and purifying the active ingredients by means of AB-8 macroporous adsorption resin technique, which is a simple process with low costs as the resin is reusable, thus being suitable for industry production. Moreover, in embodiments of the present disclosure, an optimal condition has been selected by carefully investigating corresponding parameters, which is verified in a pilot test and can be transited into industrial production, thereby increasing the content of the active ingredient. In the total flavonoids extract of Desmodium Styracifolium, the total flavonoids of Desmodium styracifolium is of a content between 50% and 80%, in which a schaftoside content is between 3.0% and 12.0%.

4. In a formulation process of the present disclosure, total flavonoids of Desmodium Styracifolium is dispersed in the hydrophilic carrier material by the solid dispersion technique and adding a certain amount of surfactant, which improves wettability of the drug, ensures its high dispersion, and improves its dissolution and absorption. The dissolution and absorption of total flavonoids of Desmodium Styracifolium can be promoted to the maximum extent when the hydrophilic carrier material of the solid disperdion is a combination of povidone K₃₀ and poloxamer 188 (a weight ratio of povidone K₃₀ to poloxamer 188 is 2:1) and the surfactant is added (a weight ratio of hydrophilic carrier of the solid disperdion to the surfactant is 10:1). Moreover, an optimal formula for preparing the solid dispersion containing total flavonoids of Desmodium Styracifolium is determined to be of total flavonoids of Desmodium Styracifolium: povidone K₃₀: poloxamer 188: sodium dodecyl sulfate=1:4:2:0.6 (by weight), with which the total flavonoids of Desmodium Styracifolium have a good dissolution. The oral solid formulation containing total flavonoids of Desmodium Styracifolium obtained by the present disclosure has a dissolution rate which can be stabilized between 88% and 92% and has small differences among different batches, with the solid dispersion technology which effectively ensures the quality of the products, improves the stability of the formulation containing effective components, has a simple process and a high operability, and is completely suitable for a large scale of industry production.

5. As compared with commercially available medicaments with the same use, the oral solid formulation containing total flavonoids of Desmodium Styracifolium prepared according to embodiments of the present disclosure has a developed production process, a clear active ingredient basis, a controllable quality, a clear and definite clinical indication, a significant pharmacological efficacy, a small dosage, a safe and convenient administration and a mild side effect, so that the capsule of the present disclosure has the advantage of being suitable to technique and quality standard of the modern manufacturing industry. The capsule is mainly used to treat: dampness-heat, diuresis and expelling stone, a dribbling pain caused by stagnation of dampness-heat and urinary stone.

Additional aspects and advantages of embodiments of present disclosure will be given in part in the following descriptions, become apparent in part from the following descriptions, or be learned from the practice of the embodiments of the present disclosure.

### BRIEF DESCRIPTION OF THE DRAWINGS

These and other aspects and advantages of embodiments of the present disclosure will become apparent and more readily appreciated from the following descriptions made with reference to the drawings, in which:

FIG. 1 is a leaking curve showing an amount of total flavonoids of Desmodium Styracifolium in a sample solution adsorbed by an AB-8 macroporous resin column according to an embodiment of the present disclosure;

FIG. 2 is an eluting curve showing an amount of total flavonoids of Desmodium Styracifolium eluted by the resin column with 60% ethanol as an eluent according to an embodiment of the present disclosure; and

FIG. 3 is a curve for comparing the dissolutions in vitro of a tablet containing total flavonoids of Desmodium Styracifolium prepared with the solid dispersion process (embodiment 20) and that prepared with a common wet granulation process (formula 1) according to a comparative embodiment.

### DETAILED DESCRIPTION

In the following, embodiments of the present disclosure will be described in detail, whose examples will be shown in drawings. Embodiments described in the following with reference to drawings are explanatory, illustrative, and used to generally understand the present disclosure, and shall not be construed to limit the present disclosure.

### Embodiment 1

### Preparation of Total Flavonoids of Desmodium Styracifolium

(1) Extracting method: free flavonoids and flavonoid glycosides generally can be extracted with an organic solvent according to the solubility of flavonoids, for example, ethanol with relative high concentration is commonly used in industrial production. Based on common industrialized method for extracting flavonoids, the extraction in the present study is performed with ethanol having a concentration of 60% to 95% (as an extraction solvent) twice, which is more economical and practical according to conventional production method.

Study on extraction process of refluxing with ethanol: Parameters for the extraction process, including ethanol concentration, ethanol weight (with respect to the weight of raw material of Desmodium Styracifolium), and extraction period, were determined by L9 (34) orthogonal tests, which took the total flavonoids of Desmodium Styracifolium as index. A content of the total flavonoids in an extract is measured by UV-visible spectrophotometry, and comparative analysis was performed with the content of total flavonoids and a net weight of total flavonoids in a dry extract as evaluation indexes. Factors and levels of experiments were designed as shown in Table 1, and analysis results are shown in Table 2.

### Table 1 Factors and Levels of Experiments

| | Factors | | | | | |
|---|---|---|---|---|---|---|
| | | B | | | | |
| | | Ethanol amount (with | | | | |
| | A | respect to the weight of raw | | | | |
| | Ethanol | material of Desmodium | | | D | |
| | concentration | Styracifolium) | | C | extraction period (h) | |
| Levels | (%) | 1^{st} time | 2^{nd} time | Control | 1^{st} time | 2^{nd} time |
| 1 | 60 | 8 | 6 | — | 1.0 | 1.0 |
| 2 | 80 | 10 | 8 | — | 1.5 | 1.0 |
| 3 | 95 | 12 | 10 | — | 2.0 | 1.5 |

### Table 2 Orthogonal Tests and Analysis Results

| | | | | | Content | Net weight |
|---|---|---|---|---|---|---|
| | | | | | of total | of total |
| Experiment | | | | | flavonoids | flavonoids |
| No. | A | B | C | D | (%) | (g) |
| 1 | 1 | 1 | 1 | 1 | 19.0 | 0.67 |
| 2 | 1 | 2 | 2 | 2 | 23.8 | 1.01 |
| 3 | 1 | 3 | 3 | 3 | 23.0 | 1.11 |
| 4 | 2 | 1 | 2 | 3 | 27.4 | 1.05 |
| 5 | 2 | 2 | 3 | 1 | 25.5 | 1.03 |
| 6 | 2 | 3 | 1 | 2 | 30.7 | 1.15 |
| 7 | 3 | 1 | 3 | 2 | 15.0 | 0.45 |
| 8 | 3 | 2 | 1 | 3 | 16.0 | 0.48 |
| 9 | 3 | 3 | 2 | 1 | 16.8 | 0.49 |
| K1 | 2.790 | 2.170 | 2.300 | 2.190 | | |
| K2 | 3.230 | 2.520 | 2.550 | 2.610 | | |
| K3 | 1.420 | 2.750 | 2.590 | 2.640 | | |
| k1 | 0.930 | 0.723 | 0.767 | 0.730 | | |
| k2 | 1.077 | 0.840 | 0.850 | 0.870 | | |
| k3 | 0.473 | 0.917 | 0.863 | 0.880 | | |
| R | 0.604 | 0.194 | 0.096 | 0.150 | | |

Visual analysis: As can be seen from R values shown in Table 2, R_{A}>R_{B}>R_{D}, which shows that an order of factors is A>B>D. And as can be seen from k values, A2>A1>A3, B3>B2>B1, D3>D2>D1, therefore, an optimal level combination of factors is A2B3D3. That is, an optimum extraction process for total flavonoids of Desmodium Styracifolium is: extracting twice with 80% ethanol, having a weight of 12 times as heavy as the raw material of Desmodium styracifolium for 2 hours for the first time, and having a weight of 10 times as heavy as the raw material of Desmodium styracifolium for 1.5 hours for the second time.

(2) Study on Purification Process with an Extracting Solution of Desmodium Styracifolium Obtained Through Above Optimized Extraction Conditions by Adsorption onto a Macroporous Resin Column:

1) Study on Screening Macroporous Resins

Resin: AB-8 resin (Nankai University), D101 resin (Shandong Lukang), HPD100 resin (Hebei Baocang Co., Ltd).

a) Studies on Static Saturated Adsorption and Desorption Elution with Total Flavonoids in the Sample Solution Using Different Macroporous Adsorption Resins

2 g well-treated (pumping filtrated till without water drop) macroporous resin was weighted precisely, and added into 100 mL ground erlenmeyer flask, followed by precisely added with 50 mL sample solution and then continuously shaken 24 hours on an oscillator for fully adsorption. The resulting supernatants was measured with the concentrations of total flavonoids therein. The saturated adsorption capacity of the resin was calculated by the following formula: saturated adsorption capacity=[(initial concentration-concentration after adsorption)×volume of adsorption solution]/resin weight], elution rate=(eluent concentration×elution volume)/saturated adsorption capacity×100%, and the results are shown in Table 3.

Table 3 results of static saturated adsorption and desorption as for three types of resin

Total flavonoids

| | Saturated adsorption | Elution rate |
|---|---|---|
| Resin type | capacity (mg/g) | (%) |
| AB-8 | 58.36 | 89.75 |
| D101 | 49.27 | 81.23 |
| HPD100 | 52.41 | 85.35 |

The results show that, saturated adsorption capacities, elution volumes and elution rates of AB-8, D101, HPD 100 types of macroporous resins for total flavonoids in the sample solution are relatively close in the static adsorption and desorption tests, and static adsorption and desorption properties of the three types of macroporous resins were further investigated.

b) Study on Static Saturated Adsorption and Desorption Properties with Total Flavonoids in the Sample Solution Using the Three Types of Macroporous Adsorption Resins

For three types of macroporous resins, 2 g each type of macroporous resins was weighed precisely and packed into a column for use. 100 mL loading sample was subjected to adsorption onto respective macroporous resin column at a flow rate of 1 mL/min thereby obtaining first effluents, which was subjected to adsorption again with the same column thereby obtaining second effluents. Each macroporous resin column adsorbed with loading sample was eluted with a certain volume of water to obtain an eluted solution, which was used for measuring the content of total flavonoids in the eluted solution. The adsorption ratio of respective macroporous resin was calculated based on the following formula: adsorption ratio=[(a substance content in the loading sample—a substance content in the second effluent−a substance content in the eluted solution)]/filled resin weight], elution ratio=[concentration of the eluted solution×volume of the eluted solution/filled resin weight], and the results are shown in Table 4.

Table 4 results of saturated adsorption on and desorption on of three types of resin

Total flavonoids

| Resin type | Adsorption ratio (mg/g) | Elution ratio (%) |
|---|---|---|
| AB-8 | 50.24 | 86.52 |
| D101 | 40.78 | 78.34 |
| HPD100 | 45.02 | 80.19 |

The results show that, the AB-8 macroporous resin is of good adsorption ratio and the elution ratio of total flavonoids of Desmodium Styracifolium, and high safety, which has been most applied in domestic pharmaceutical manufacturing industry, therefore, the present study selected AB-8 macroporous resin to purify the total flavonoids of Desmodium Styracifolium.

2) Study on Purification Process with AB-8 Macroporous Resin

a) Optimization to Adsorption Conditions

L9 (34) orthogonal table was used to design levels and factors including a concentration of the loading sample (by the weight of raw material contained in the loading sample), an adsorption velocity and a ratio of diameter to height, as shown in Table 5. Contents of the total flavonoids were measured for the following 9 testing groups, adsorption ratios were calculated, and comprehensive evaluation was performed. Analysis results are shown in Table 6.

TABLE 5
Table of factors and levels

| | | B | | |
|---|---|---|---|---|
| | A | adsorption | | |
| | concentration | velocity | | |
| | of | (multiples | | D |
| | loading sample | of column | C | ratio of diameter |
| Levels\Factors | (g/mL) | volume/h) | Control | to height |
| 1 | 0.1 | 2 | — | 1:4 |
| 2 | 0.2 | 4 | — | 1:8 |
| 3 | 0.4 | 6 | — | 1:12 |

### Table 6 Orthogonal Design and Analysis Results

| | | | | | Content | Net weight |
|---|---|---|---|---|---|---|
| | | | | | of total | of total |
| Experiment | | | | | flavonoids | flavonoids |
| NO. | A | B | C | D | (%) | (g) |
| 1 | 1 | 1 | 1 | 1 | 70.35 | 0.28 |
| 2 | 1 | 2 | 2 | 2 | 66.75 | 0.22 |
| 3 | 1 | 3 | 3 | 3 | 63.08 | 0.26 |
| 4 | 2 | 1 | 2 | 3 | 69.21 | 0.25 |
| 5 | 2 | 2 | 3 | 1 | 66.06 | 0.24 |
| 6 | 2 | 3 | 1 | 2 | 68.54 | 0.28 |
| 7 | 3 | 1 | 3 | 2 | 60.97 | 0.27 |
| 8 | 3 | 2 | 1 | 3 | 61.21 | 0.24 |
| 9 | 3 | 3 | 2 | 1 | 66.09 | 0.20 |
| K1 | 0.760 | 0.800 | 0.800 | 0.720 | | |
| K2 | 0.770 | 0.700 | 0.670 | 0.770 | | |
| K3 | 0.710 | 0.740 | 0.770 | 0.750 | | |
| k1 | 0.253 | 0.267 | 0.267 | 0.240 | | |
| k2 | 0.257 | 0.233 | 0.223 | 0.257 | | |
| k3 | 0.237 | 0.247 | 0.257 | 0.250 | | |
| R | 0.020 | 0.034 | 0.044 | 0.017 | | |

Result analysis: As can be seen from R values shown in Table 2, RB>RA>RD, which shows that an order of factors was B>A>D. And as can be seen from k values, A2>A1>A3, B1>B2>B2, D2>D3>D1, therefore, an optimal level combination of factors was A2B13D2. Therefore, an optimum adsorption condition of total flavonoids alternatively is: 0.2 g/ml of loading sample, 2 BV/h of adsorption velocity, and 1:8 of ratio of diameter to height.

b) Investigation in a Volume of Loading Sample

0.2 g/mL loading sample was applied onto 20 g AB-8 resin column (400 mm×20 mm) at the flow rate of 2 Bv/h. 10 mL was collected from each fraction. Concentrations of total flavonoids in each fraction were measured and calculated, and a leaking curve was plotted, the results are shown in FIG. 1. As can be seen from the figure, total flavonoids begin to leak when the volume of the loading sample was 50 mL (i.e. 10 g raw material of Desmodium Styracifolium), and an adsorption saturation state was reached when the volume of the loading sample was 600 mL (about 30 times the weight of filled resin).

c) Investigation in Washing Condition

50 mL loading sample was subjected to adsorption in accordance above optimum adsorption condition, purified with water, with every 20 mL of effluent for one fraction, inspection was performed through α-naphthol reaction, at the same time, weight of dry extract was determined, α-naphthol reaction was negative and the weight of dry extract does not change anymore after washing with 300 mL water, indicating that sugars adsorbed by the resin column can be substantially removed after washing with 300 mL water (about 15 times the weight of resin).

d) Investigation in Ethanol Concentration for Elution

Another five copies of resin (with 20 g for each copy) each were subjected to adsorptions, purified in accordance above adsorption condition and washing condition followed by separately eluted with 400 mL ethanol each having a concentration of 30%, 45%, 60%, 75%, 90% at same flow rate, the content and desorption rate of total flavonoids were determined and calculated, results are shown in Table 7.

### Table 7 Results of Ethanol Concentration for Elution of Total Flavonoids of Desmodium Styracifolium

| | Ethanol concentration (%) | | | | |
|---|---|---|---|---|---|
| | 30 | 45 | 60 | 75 | 90 |
| Desorption rate (%) | 38.16 | 59.25 | 85.63 | 81.56 | 79.21 |
| Weight of dry extract (mg) | 87.23 | 178.5 | 231.6 | 216.2 | 209.3 |
| Content of total flavonoids | 35.21 | 42.06 | 58.63 | 55.45 | 56.82 |
| (%) | | | | | |

The above results show that, both the desorption rate and content of total flavonoids are high when the ethanol concentration is 60% or more, and desorption capacities are considerable when the ethanol concentrations separately are 60%, 75% and 90%, the present test chose 60% ethanol as the elution solvent for the sake of manufacturing cost.

e) Investigation in Elution Velocity

Dynamic adsorption was performed with 60% ethanol as the elution solvent at flow rate of 1, 3, 5 column bed volumes per hour, respectively, according to above conditions, ethanol eluents were collected, the content and desorption rate of total flavonoids were determined and calculated, results are shown in Table 8.

### Table 8 Results of Elution Velocity of Ethanol for Elution of Total Flavonoids of Desmodium Styracifolium

| | | Content of total flavonoids in |
|---|---|---|
| Elution velocity | Desorption rate (%) | dry extract (%) |
| 1BV/h | 88.31 | 57.92 |
| 3BV/h | 87.45 | 56.37 |
| 5BV/h | 80.16 | 50.08 |

Results show that, there was no big difference between elution velocity of 1 column bed volume per hour and elution velocity of 3 column bed volumes per hour, and choosing elution velocity of 3 BV/h was more reasonable, considering the production efficiency.

f) Investigation in Elution Volume

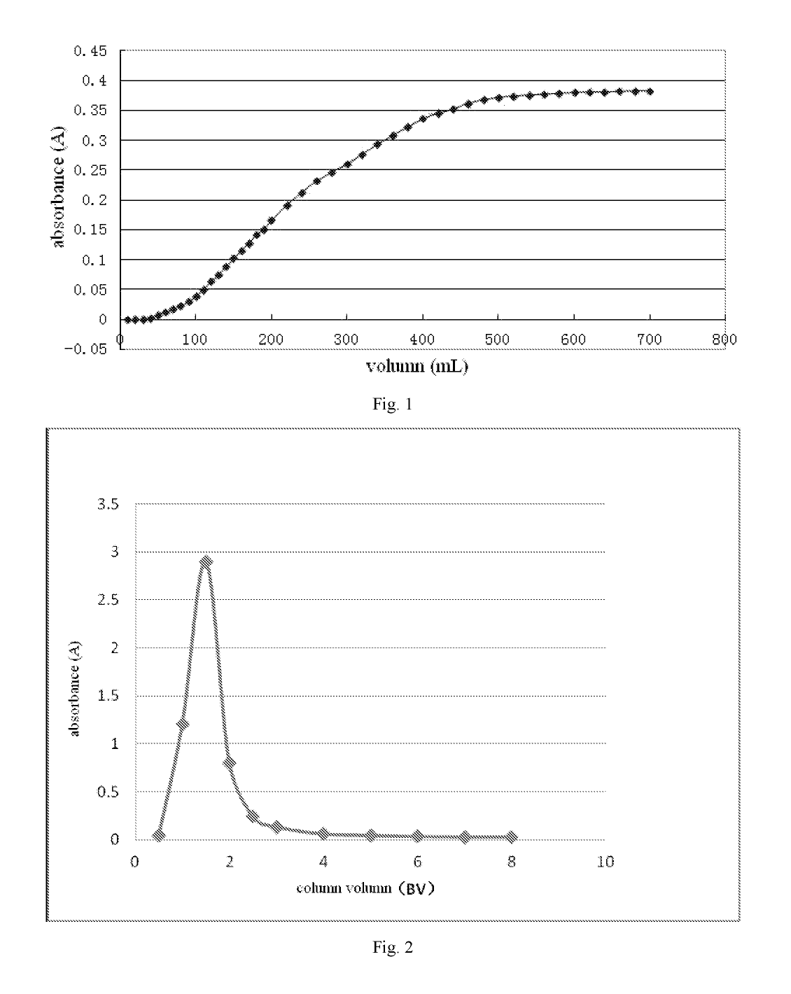

Dynamic adsorption was performed with 60% ethanol as elution solvent according to above conditions, the effluent was quantitative collected and in which the content of total flavonoids were determined. Results are shown in FIG. 2. Results show that, flavonoids adsorbed by 20 g resin can be completely eluted with 240 mL (8 times the resin column volume) of 60% ethanol. Therefore, flavonoids adsorbed by 20 g resin can be completely eluted with 240 mL (8 times the resin column volume) of 60% ethanol at 3 BV/h of elution velocity.

### Embodiment 2

### Preparation of Total Flavonoids of Desmodium Styracifolium

50 g raw material of Desmodium Styracifolium was weighted, heated and refluxed at a temperature of 55° C. for 2 hours for first extraction with 80% ethanol having a weight of 12 times as heavy as the raw material, and then heated and refluxed at a temperature of 55° C. for 1.5 hours for second extraction with 80% ethanol having a weight of 10 times as heavy as the raw material followed by mixing. The alcohol extraction solution was concentrated to be of a volume 5 times the weight of the raw material followed by still standing and filtering, thereby obtaining a filtrate (i.e., loading sample) for use. 100 g AB-8 macroporous resin (in pharmaceutical grade) was immersed into a suitable amount of ethanol, and then packed into a column via a wet method followed by treatment for use. The filtrate (the loading sample) was subjected to adsorption onto an AB-8 macroporous resin column at a flow rate of 2 column bed volumes per hour, eluted and purified with water having a volume of 10 times the weight of filled resin, then eluted with 60% ethanol having a volume of 8 column bed volumes at a flow rate of 2 column bed volumes per hour, to obtain an eluted solution. The eluted solution was concentrated to recycle ethanol and to obtain a concentrated solution with a relative density of 1.22, then dried under reduced pressure at a temperature of 75° C. and smashing to obtain 1.10 g total flavonoids extract of Desmodium Styracifolium.

### Embodiment 3

### Preparation of Total Flavonoids of Desmodium Styracifolium

200 g raw material of Desmodium Styracifolium was weighted, heated and refluxed at a temperature of 55° C. for 2 hours for first extraction with 80% ethanol having a weight of 12 times as heavy as the raw material, and then heated and refluxed at a temperature of 55° C. for 1.5 hours for second extraction with 80% ethanol having a weight of 10 times as heavy as the raw material followed by mixing. The alcohol extraction solution was concentrated to be of a volume 5 times the weight of the raw material followed by still standing and filtering, thereby obtaining a filtrate (i.e., loading sample) for use. 400 g AB-8 macroporous resin (in pharmaceutical grade) was immersed into a suitable amount of ethanol, and then packed into a column via a wet method followed by treatment for use. The filtrate (the loading sample) was subjected to adsorption onto an AB-8 macroporous resin column at a flow rate of 2 column bed volumes per hour, eluted and purified with water having a volume of 10 times the weight of filled resin, then eluted with 60% ethanol having a volume of 8 column bed volumes at a flow rate of 2 column bed volumes per hour, to obtain an eluted solution. The eluted solution was concentrated to recycle ethanol and to obtain a concentrated solution with a relative density of 1.22, then dried under reduced pressure at a temperature of 75° C. and smashing to obtain 4.03 g total flavonoids extract of Desmodium Styracifolium (placed in a shady and cool place).

By means of UV-visible spectrophotometry, the content of the total flavonoids was measured to be of 63.31% (by dried product, %), the content of schaftoside was 5.38% (by dried product, %).

### Embodiment 4

### Preparation of Total Flavonoids of Desmodium Styracifolium

200 g raw material of Desmodium Styracifolium was weighted, heated and refluxed at a temperature of 60° C. for 3 hours for first extraction with 95% ethanol having a weight of 14 times as heavy as the raw material, and heated and refluxed at a temperature of 50° C. for 2 hours for second extraction with 95% ethanol having a weight of 12 times as heavy as the raw material, then heated and refluxed at a temperature of 50° C. for 1 hour for third extraction with 80% ethanol having a weight of 8 times as heavy as the raw material followed by mixing. The alcohol extraction solution was concentrated to be of a volume 8 times the weight of the raw material followed by still standing and filtering, thereby obtaining a filtrate (i.e., loading sample) for use. 400 g AB-8 macroporous resin (in pharmaceutical grade) was immersed into a suitable amount of ethanol, and then packed into a column via a wet method followed by treatment for use.

The filtrate (the loading sample) was subjected to adsorption onto an AB-8 macroporous resin column at a flow rate of 2 column bed volumes per hour, eluted and purified with water having a volume of 12 times the weight of filled resin, then eluted with 95% ethanol having a volume of 10 column bed volumes at a flow rate of 2 column bed volumes per hour, to obtain an eluted solution. The eluted solution was concentrated to recycle ethanol and to obtain a concentrated solution with a relative density of 1.10, then dried under reduced pressure at a temperature of 75° C. and smashing to obtain 4.03 g total flavonoids extract of Desmodium Styracifolium (placed in a shady and cool place). By means of UV-visible spectrophotometry, the content of the total flavonoids was measured to be of 71.65% (by dried product, %), and the content of schaftoside was 10.30% (by dried product, %).

### Embodiment 5

### Preparation of Total Flavonoids of Desmodium Styracifolium

200 g raw material of Desmodium Styracifolium was weighted, heated and refluxed at a temperature of 55° C. for 2 hours for first extraction with 70% ethanol having a weight of 12 times as heavy as the raw material, and then heated and refluxed at a temperature of 55° C. for 1.5 hours for second extraction with 70% ethanol having a weight of 10 times as heavy as the raw material followed by mixing. The alcohol extraction solution was concentrated to be of a volume 5 times the weight of the raw material followed by still standing and filtering, thereby obtaining a filtrate (i.e., loading sample) for use. 400 g AB-8 macroporous resin (in pharmaceutical grade) was immersed into a suitable amount of ethanol, and then packed into a column via a wet method followed by treatment for use.

The filtrate (the loading sample) was subjected to adsorption onto an AB-8 macroporous resin column at a flow rate of 1 column bed volumes per hour, eluted and purified with water having a volume of 10 times the weight of filled resin, then eluted with 60% ethanol having a volume of 8 column bed volumes at a flow rate of 2 column bed volumes per hour, to obtain an eluted solution. The eluted solution was concentrated to recycle ethanol and to obtain a concentrated solution with a relative density of 1.22, then dried under reduced pressure at a temperature of 75° C. and smashing to obtain 4.68 g total flavonoids extract of Desmodium Styracifolium (placed in a shady and cool place). By means of UV-visible spectrophotometry, the content of the total flavonoids was measured to be of 60.07% (by dried product, %), the content of schaftoside was 4.45% (by dried product, %).

### Embodiment 6

### Preparation of Total Flavonoids of Desmodium Styracifolium

200 g raw material of Desmodium Styracifolium was weighted, heated and refluxed for extraction at a temperature of 60° C. for 3 hours with 50% ethanol having a weight of 12 times as heavy as the raw material to obtain an alcohol extraction solution. The alcohol extraction solution was concentrated to be of a volume 2 times the weight of the raw material followed by still standing and filtering, thereby obtaining a filtrate (i.e., loading sample) for use. 400 g AB-8 macroporous resin (in pharmaceutical grade) was immersed into a suitable amount of ethanol, and then packed into a column via a wet method followed by treatment for use.

The filtrate (the loading sample) was subjected to adsorption onto an AB-8 macroporous resin column at a flow rate of 2 column bed volumes per hour, eluted and purified with water having a volume of 8 times the weight of filled resin, then eluted with 40% ethanol having a volume of 6 column bed volumes at a flow rate of 2 column bed volumes per hour, to obtain an eluted solution. The eluted solution was concentrated to recycle ethanol and to obtain a concentrated solution with a relative density of 1.30, then dried under reduced pressure at a temperature of 75° C. and smashing to obtain 3.89 g total flavonoids extract of Desmodium Styracifolium (placed in a shady and cool place). By means of UV-visible spectrophotometry, the content of the total flavonoids was measured to be of 52.64% (by dried product, %), the content of schaftoside was 4.17% (by dried product, %).

### Embodiment 7

### Preparation of Total Flavonoids of Desmodium Styracifolium

50 kg raw material of Desmodium Styracifolium was weighted, heated and refluxed at a temperature of 55° C. for 2 hours for first extraction with 80% ethanol having a weight of 12 times as heavy as the raw material, and then heated and refluxed at a temperature of 55° C. for 1.5 hours for second extraction with 80% ethanol having a weight of 10 times as heavy as the raw material followed by mixing. The alcohol extraction solution was concentrated to be of a volume 5 times the weight of the raw material followed by still standing and filtering, thereby obtaining a filtrate (i.e., loading sample) for use. 100 kg AB-8 macroporous resin (in pharmaceutical grade) was immersed into a suitable amount of ethanol, and then packed into a column via a wet method followed by treatment for use.

The filtrate (the loading sample) was subjected to adsorption onto an AB-8 macroporous resin column at a flow rate of 2 column bed volumes per hour, eluted and purified with water having a volume of 10 times the weight of filled resin, then eluted with 60% ethanol having a volume of 8 column bed volumes at a flow rate of 2 column bed volumes per hour, to obtain an eluted solution. The eluted solution was concentrated to recycle ethanol and to obtain a concentrated solution with a relative density of 1.22, then dried under reduced pressure at a temperature of 75° C. and smashing to obtain 1.12 kg total flavonoids extract of Desmodium Styracifolium (placed in a shady and cool place). By means of UV-visible spectrophotometry, the content of the total flavonoids was measured to be of 59.49% (by dried product, %), the content of schaftoside was 5.10% (by dried product, %).

### Embodiment 8

### Preparation of Total Flavonoids of Desmodium Styracifolium

50 kg raw material of Desmodium Styracifolium was weighted, heated and refluxed at a temperature of 55° C. for 2 hours for first extraction with 80% ethanol having a weight of 12 times as heavy as the raw material, and then heated and refluxed at a temperature of 55° C. for 1.5 hours for second extraction with 80% ethanol having a weight of 10 times as heavy as the raw material followed by mixing. The alcohol extraction solution was concentrated to be of a volume 5 times the weight of the raw material followed by still standing and filtering, thereby obtaining a filtrate (i.e., loading sample) for use. 100 kg AB-8 macroporous resin (in pharmaceutical grade) was immersed into a suitable amount of ethanol, and then packed into a column via a wet method followed by treatment for use.

The filtrate (the loading sample) was subjected to adsorption onto an AB-8 macroporous resin column at a flow rate of 2 column bed volumes per hour, eluted and purified with water having a volume of 10 times the weight of filled resin, then eluted with 60% ethanol having a volume of 8 column bed volumes at a flow rate of 2 column bed volumes per hour, to obtain an eluted solution. The eluted solution was concentrated to recycle ethanol and to obtain a concentrated solution with a relative density of 1.22, then dried under reduced pressure at a temperature of 75° C. and smashing to obtain 1.14 kg total flavonoids extract of Desmodium Styracifolium (placed in a shady and cool place). By means of UV-visible spectrophotometry, the content of the total flavonoids was measured to be of 59.37% (by dried product, %), the content of schaftoside was 5.01% (by dried product, %).

Results show that: the process parameters studied by the present experiments was feasible and can be used in industrial production.

### Embodiment 9

### Preparation of Tablets Containing Total Flavonoids of Desmodium Styracifolium

Formula:

| | | |
|---|---|---|
| Total flavonoids of Desmodium Styracifolium | 50 | g |
| Povidone K₃₀ | 200 | g |
| Poloxamer 188 | 100 | g |
| Sodium dodecyl sulfate | 30 | g |
| Lactose | 50 | g |
| Sodium croscarmellose | 20 | g |
| Sodium stearyl fumarate | 5 | g |
| Total | 1000 | tablets |

Preparation was performed by the following steps:

a. preparing total flavonoids of Desmodium Styracifolium according to Embodiment 8;

b. preparing a solid dispersion containing total flavonoids of Desmodium Styracifolium: dissolving 50 g total flavonoids of Desmodium Styracifolium, 200 g povidone K₃₀, 100 g poloxamer 188 and 30 g sodium dodecyl sulfate in respective formula dosage, sieved at 80 meshes in advance respectively, with 50% ethanol under stirring and at a temperature of 65° C., followed by removing the solvent via evaporation under reduced pressure at a temperature of 50° C., vacuum drying at a temperature of 40° C., smashing and sieving at 80 meshes, so as to obtain the solid dispersion containing total flavonoids of Desmodium Styracifolium;

c. preparing granules containing total flavonoids of Desmodium Styracifolium: mixing 50 g lactose and 20 g sodium croscarmellose, sieved at 80 meshes in advance respectively, with the solid dispersion containing the total flavonoids of Desmodium Styracifolium obtained in step (b) to be uniform, followed by preparing a soften material with suitable amount of water, granulating at 20 meshes, drying at a temperature of 55° C., size stabilizing, and mixing with 5 g sodium stearyl fumarate to be uniform, so as to obtain the granules containing total flavonoids of Desmodium Styracifolium;

d. tableting: tableting the granules containing total flavonoids of Desmodium Styracifolium with a tableting machine to obtain a tablet containing total flavonoids of Desmodium Styracifolium with a dissolution rate measured to be 90.7%.

Method for Determining the Dissolution Rate:

Dissolution rate was measured in accordance with the First Method in appendix XC, part II, Chinese Pharmacopoeia, 2010 edition.

Testing sample solutions were obtained by the following steps: dissolving the above-obtained tablet in 1000 ml water (as a dissolution medium) contained in a beaker; setting a rotation rate of dissolution rate analysis instruments to be 100 rpm per min; taking 10 ml from the beaker after 5, 15, 25, 35, 45 and 60 min from operations specified in the First Method, followed by filtration; taking 1 ml secondary filtrate precisely each into a 5 ml volumetric flask and adding 0.1 M hydrochloric acid up to graduation on the volumetric flask, followed by shaken to be uniform, thereby obtaining the testing sample solutions.

Reference sample solutions were obtained by the following steps: weighing a certain amount of schaftosides precisely as a reference sample; dissolving the reference sample with an appropriate volume of ethanol in a volumetric flask; adding 0.1 M hydrochloric acid up to graduation on the volumetric flask, thereby obtaining the reference sample solutions each having a schaftoside concentration of 15 μg/ml.

The testing sample solutions and the reference sample solutions were subjected to ultraviolet spectrophotometry (appendix IV A) at 270 nm, for calculating the dissolution rate of each tablet.

### Embodiment 10

### Preparation of Tablets Containing Total Flavonoids of Desmodium Styracifolium

Formula:

| | | |
|---|---|---|
| Total flavonoids of Desmodium Styracifolium | 33 | g |
| Povidone K₃₀ | 132 | g |
| Poloxamer 188 | 66 | g |
| Sodium dodecyl sulfate | 6.6 | g |
| Lactose | 50 | g |
| Cross-linked povidone | 20 | g |
| Aerosil | 1 | g |
| Total | 1000 | tablets |

Preparation: identical to Embodiment 9.

### Embodiment 11

### Preparation of Tablets Containing Total Flavonoids of Desmodium Styracifolium

Formula:

| | | |
|---|---|---|
| Total flavonoids of Desmodium Styracifolium | 33 | g |
| Povidone K₃₀ | 132 | g |
| Poloxamer 188 | 66 | g |
| Sodium dodecyl sulfate | 19.8 | g |
| Lactose | 30 | g |
| Sodium croscarmellose | 20 | g |
| Sodium stearyl fumarate | 3 | g |
| Total | 1000 | tablets |

Preparation: identical to Embodiment 9.

### Embodiment 12

### Preparation of Tablets Containing Total Flavonoids of Desmodium Styracifolium

Formula:

| | | |
|---|---|---|
| Total flavonoids of Desmodium Styracifolium | 33 | g |
| Povidone K₃₀ | 132 | g |
| Poloxamer 188 | 66 | g |
| Sodium dodecyl sulfate | 33 | g |
| Microcrystalline cellulose | 10 | g |
| Sodium croscarmellose | 20 | g |
| Magnesium stearate | 2 | g |
| Total | 1000 | tablets |

Preparation: identical to Embodiment 9

### Embodiment 13

### Preparation of Tablets Containing Total Flavonoids of Desmodium Styracifolium

Formula:

| | | |
|---|---|---|
| Total flavonoids of Desmodium Styracifolium | 33 | g |
| Povidone K₃₀ | 132 | g |
| Poloxamer 188 | 66 | g |
| Tween 80 | 19.8 | g |
| Lactose | 40 | g |
| Sodium carboxymethyl starch | 5 | g |
| Sodium stearyl fumarate | 4 | g |
| Total | 1000 | tablets |

Preparation: identical to Embodiment 9. Dissolution ratio is determined to be 90.4%.

### Embodiment 14

### Preparation of Tablets Containing Total Flavonoids of Desmodium Styracifolium

Formula:

| | | |
|---|---|---|
| Total flavonoids of Desmodium Styracifolium | 33 | g |
| Povidone K₃₀ | 132 | g |
| Poloxamer 188 | 66 | g |
| Methoxypolyethylene glycol | 19.8 | g |
| Microcrystalline cellulose | 20 | g |
| Sodium carboxymethyl starch | 15 | g |
| Sodium stearyl fumarate | 3 | g |
| Total | 1000 | tablets |

Preparation: identical to Embodiment 9.

### Embodiment 15

### Preparation of Tablets Containing Total Flavonoids of Desmodium Styracifolium

Formula:

| | | |
|---|---|---|
| Total flavonoids of Desmodium Styracifolium | 50 | g |
| Povidone K₃₀ | 200 | g |
| Poloxamer 188 | 100 | g |
| Sodium dodecyl sulfate | 10 | g |
| Lactose | 50 | g |
| Sodium croscarmellose | 20 | g |
| Sodium stearyl fumarate | 5 | g |
| Total | 1000 | tablets |

Preparation: identical to Embodiment 9

### Embodiment 16

### Preparation of Tablets Containing Total Flavonoids of Desmodium Styracifolium

Formula:

| | | |
|---|---|---|
| Total flavonoids of Desmodium Styracifolium | 50 | g |
| Povidone K₃₀ | 200 | g |
| Poloxamer 188 | 100 | g |
| Sodium dodecyl sulfate | 50 | g |
| Lactose | 50 | g |
| Sodium croscarmellose | 20 | g |
| Sodium stearyl fumarate | 5 | g |
| Total | 1000 | tablets |

Preparation: identical to Embodiment 9

### Embodiment 17

### Preparation of Tablets Containing Total Flavonoids of Desmodium Styracifolium

Formula:

| | | |
|---|---|---|
| Total flavonoids of Desmodium Styracifolium | 50 | g |
| Povidone K₃₀ | 200 | g |
| Poloxamer 188 | 100 | g |
| Tween 80 | 30 | g |
| Lactose | 50 | g |
| Sodium croscarmellose | 20 | g |
| Sodium stearyl fumarate | 5 | g |
| Total | 1000 | tablets |

Preparation: identical to Embodiment 9

### Embodiment 18

### Preparation of tablets containing total flavonoids of Desmodium Styracifolium

Formula

| | | |
|---|---|---|
| Total flavonoids of Desmodium Styracifolium | 50 | g |
| Povidone K₃₀ | 200 | g |
| Poloxamer 188 | 100 | g |
| Methoxypolyethylene glycol | 30 | g |
| Lactose | 50 | g |
| Sodium croscarmellose | 20 | g |
| Sodium stearyl fumarate | 5 | g |
| Total | 1000 | tablets |

Preparation: identical to Embodiment 9. Dissolution ratio is determined to be 91.1%.

### Embodiment 19

### Preparation of Tablets Containing Total Flavonoids of Desmodium Styracifolium

Formula:

| | | |
|---|---|---|
| Total flavonoids of Desmodium Styracifolium | 50 | g |
| Povidone K₃₀ | 200 | g |
| Poloxamer 188 | 100 | g |
| Sodium dodecyl sulfate | 30 | g |
| Pregelatinized starch | 50 | g |
| Sodium croscarmellose | 20 | g |
| Sodium stearyl fumarate | 5 | g |
| Total | 1000 | tablets |

Preparation: identical to Embodiment 9

### Embodiment 20

### Preparation of Tablets Containing Total Flavonoids of Desmodium Styracifolium

Formula:

| | | |
|---|---|---|
| Total flavonoids of Desmodium Styracifolium | 50 | g |
| Povidone K₃₀ | 200 | g |
| Poloxamer 188 | 100 | g |
| Sodium dodecyl sulfate | 50 | g |
| Cross-linked povidone | 50 | g |
| Sodium croscarmellose | 20 | g |
| Sodium stearyl fumarate | 5 | g |
| Total | 1000 | tablets |

Preparation: identical to Embodiment 9

### Embodiment 21

### Preparation of Tablets Containing Total Flavonoids of Desmodium Styracifolium

Formula:

| | | |
|---|---|---|
| Total flavonoids of Desmodium Styracifolium | 50 | g |
| Povidone K₃₀ | 200 | g |
| Poloxamer 188 | 100 | g |
| Methoxypolyethylene glycol | 30 | g |
| Microcrystalline cellulose | 50 | g |
| Sodium croscarmellose | 20 | g |
| Aerosil | 5 | g |
| Total | 1000 | tablets |

Preparation: identical to Embodiment 9

### Embodiment 22

### Preparation of Tablets Containing Total Flavonoids of Desmodium Styracifolium

Formula:

| | | |
|---|---|---|
| Total flavonoids of Desmodium Styracifolium | 66.5 | g |
| Povidone K₃₀ | 266 | g |
| Poloxamer 188 | 133 | g |
| Sodium dodecyl sulfate | 13.3 | g |
| Microcrystalline cellulose | 20 | g |
| Sodium carboxymethyl starch | 10 | g |
| Sodium stearyl fumarate | 8 | g |
| Total | 1000 | tablets |

Preparation: identical to Embodiment 9

### Embodiment 23

### Preparation of Tablets Containing Total Flavonoids of Desmodium Styracifolium

Formula:

| | | |
|---|---|---|
| Total flavonoids of Desmodium Styracifolium | 66.5 | g |
| Povidone K₃₀ | 266 | g |
| Poloxamer 188 | 133 | g |
| Sodium dodecyl sulfate | 39.9 | g |
| Lactose | 10 | g |
| Sodium croscarmellose | 15 | g |
| Sodium stearyl fumarate | 6 | g |
| Total | 1000 | tablets |

Preparation: identical to Embodiment 9. Dissolution ratio is determined to be 91.5%.

### Embodiment 24

### Preparation of Tablets Containing Total Flavonoids of Desmodium Styracifolium

Formula:

| | | |
|---|---|---|
| Total flavonoids of Desmodium Styracifolium | 66.5 | g |
| Povidone K₃₀ | 266 | g |
| Poloxamer 188 | 133 | g |
| Sodium dodecyl sulfate | 66.5 | g |
| Lactose | 1 | g |
| Cross-linked povidone | 10 | g |
| Magnesium stearate | 10 | g |
| Total | 1000 | tablets |

Preparation: identical to Embodiment 9

### Embodiment 25

### Preparation of Tablets Containing Total Flavonoids of Desmodium Styracifolium

Formula:

| | | |
|---|---|---|
| Total flavonoids of Desmodium Styracifolium | 66.5 | g |
| Povidone K₃₀ | 266 | g |
| Poloxamer 188 | 133 | g |
| Tween 80 | 39.9 | g |
| Lactose | 10 | g |
| Sodium croscarmellose | 50 | g |
| Sodium stearyl fumarate | 1 | g |
| Total | 1000 | tablets |

Preparation: identical to Embodiment 9

### Embodiment 26

### Preparation of Tablets Containing Total Flavonoids of Desmodium Styracifolium

Formula:

| | | |
|---|---|---|
| Total flavonoids of Desmodium Styracifolium | 66.5 | g |
| Povidone K₃₀ | 266 | g |
| Poloxamer 188 | 133 | g |
| Methoxypolyethylene glycol | 39.9 | g |
| Pregelatinized starch | 15 | g |
| Sodium croscarmellose | 30 | g |
| Sodium stearyl fumarate | 5 | g |
| Total | 1000 | tablets |

Preparation: identical to Embodiment 9.

### Embodiment 27

### Preparation of Tablets Containing Total Flavonoids of Desmodium Styracifolium

Formula:

| | | |
|---|---|---|
| Total flavonoids of Desmodium Styracifolium | 100 | g |
| Povidone K₃₀ | 400 | g |
| Poloxamer 188 | 200 | g |
| Sodium dodecyl sulfate | 20 | g |
| Pregelatinized starch | 50 | g |
| Sodium croscarmellose | 20 | g |
| Sodium stearyl fumarate | 5 | g |
| Total | 1000 | tablets |

Preparation: identical to Embodiment 9

### Embodiment 28

### Preparation of Tablets Containing Total Flavonoids of Desmodium Styracifolium

Formula:

| | | |
|---|---|---|
| Total flavonoids of Desmodium Styracifolium | 100 | g |
| Povidone K₃₀ | 400 | g |
| Poloxamer 188 | 200 | g |
| Sodium dodecyl sulfate | 60 | g |
| Lactose | 50 | g |
| Cross-linked povidone | 40 | g |
| Sodium stearyl fumarate | 8 | g |
| Total | 1000 | tablets |

Preparation: identical to Embodiment 9

### Embodiment 29

### Preparation of Tablets Containing Total Flavonoids of Desmodium Styracifolium

Formula:

| | | |
|---|---|---|
| Total flavonoids of Desmodium Styracifolium | 100 | g |
| Povidone K₃₀ | 400 | g |
| Poloxamer 188 | 200 | g |
| Sodium dodecyl sulfate | 100 | g |
| Microcrystalline cellulose | 10 | g |
| Sodium carboxymethyl starch | 20 | g |
| Sodium stearyl fumarate | 10 | g |
| Total | 1000 | tablets |

Preparation: identical to Embodiment 9

### Embodiment 30

### Preparation of tablets containing total flavonoids of Desmodium Styracifolium

Formula:

| | | |
|---|---|---|
| Total flavonoids of Desmodium Styracifolium | 100 | g |
| Povidone K₃₀ | 400 | g |
| Poloxamer 188 | 200 | g |
| Tween 80 | 60 | g |
| Lactose | 10 | g |
| Sodium croscarmellose | 15 | g |
| Sodium stearyl fumarate | 8 | g |
| Total | 1000 | tablets |

Preparation: identical to Embodiment 9

### Embodiment 31

### Preparation of Tablets Containing Total Flavonoids of Desmodium Styracifolium

Formula:

| | | |
|---|---|---|
| Total flavonoids of Desmodium Styracifolium | 100 | g |
| Povidone K₃₀ | 400 | g |
| Poloxamer 188 | 200 | g |
| Methoxypolyethylene glycol | 60 | g |
| Lactose | 15 | g |
| Sodium croscarmellose | 10 | g |
| Sodium stearyl fumarate | 8 | g |
| Total | 1000 | tablets |

Preparation: identical to Embodiment 9

### Embodiment 32

### Preparation of Tablets Containing Total Flavonoids of Desmodium Styracifolium

Formula:

| | | |
|---|---|---|
| Total flavonoids of Desmodium Styracifolium | 133 | g |
| Povidone K₃₀ | 532 | g |
| Poloxamer 188 | 266 | g |
| Sodium dodecyl sulfate | 26.6 | g |
| Lactose | 20 | g |
| Sodium croscarmellose | 20 | g |
| Sodium stearyl fumarate | 8 | g |
| Total | 1000 | tablets |

Preparation: identical to Embodiment 9

### Embodiment 33

### Preparation of Tablets Containing Total Flavonoids of Desmodium Styracifolium

Formula:

| | | |
|---|---|---|
| Total flavonoids of Desmodium Styracifolium | 133 | g |
| Povidone K₃₀ | 532 | g |
| Poloxamer 188 | 266 | g |
| Sodium dodecyl sulfate | 79.8 | g |
| Lactose | 20 | g |
| Cross-linked povidone | 25 | g |
| Sodium stearyl fumarate | 9 | g |
| Total | 1000 | tablets |

Preparation: identical to Embodiment 9

### Embodiment 34

### Preparation of Tablets Containing Total Flavonoids of Desmodium Styracifolium

Formula:

| | | |
|---|---|---|
| Total flavonoids of Desmodium Styracifolium | 133 | g |
| Povidone K₃₀ | 532 | g |
| Poloxamer 188 | 266 | g |
| Sodium dodecyl sulfate | 133 | g |
| Microcrystalline cellulose | 1 | g |
| Sodium carboxymethyl starch | 1 | g |
| Sodium stearyl fumarate | 10 | g |
| Total | 1000 | tablets |

Preparation: identical to Embodiment 9

### Embodiment 35

### Preparation of Tablets Containing Total Flavonoids of Desmodium Styracifolium

Formula:

| | | |
|---|---|---|
| Total flavonoids of Desmodium Styracifolium | 133 | g |
| Povidone K₃₀ | 532 | g |
| Poloxamer 188 | 266 | g |
| Tween 80 | 79.8 | g |
| Lactose | 10 | g |
| Sodium croscarmellose | 10 | g |
| Magnesium stearate | 8 | g |
| Total | 1000 | tablets |

Preparation: identical to Embodiment 9

### Embodiment 36

### Preparation of Tablets Containing Total Flavonoids of Desmodium Styracifolium

Formula:

| | | |
|---|---|---|
| Total flavonoids of Desmodium Styracifolium | 133 | g |
| Povidone K₃₀ | 532 | g |
| Poloxamer 188 | 266 | g |
| Methoxypolyethylene glycol | 79.8 | g |
| Pregelatinized starch | 5 | g |
| Cross-linked povidone | 5 | g |
| Magnesium stearate | 5 | g |
| Total | 1000 | tablets |

Preparation: identical to Embodiment 9

### Embodiment 37

### Preparation of Capsules Containing Total Flavonoids of Desmodium Styracifolium

Formula:

| | | |
|---|---|---|
| Total flavonoids of Desmodium Styracifolium | 50 | g |
| Povidone K₃₀ | 200 | g |
| Poloxamer 188 | 100 | g |
| Sodium dodecyl sulfate | 30 | g |
| Lactose | 50 | g |
| Sodium croscarmellose | 20 | g |
| Sodium stearyl fumarate | 5 | g |
| Total | 1000 | capsules |

Preparation was performed by the following steps:

a. preparing total flavonoids of Desmodium Styracifolium according to Embodiment 8;

b. preparing a solid dispersion containing total flavonoids of Desmodium Styracifolium: dissolving 50 g total flavonoids of Desmodium Styracifolium, 200 g povidone K₃₀, 100 g poloxamer 188 and 30 g sodium dodecyl sulfate in respective formula dosage, sieved at 80 meshes in advance respectively, with 50% ethanol under stirring and at a temperature of 65° C., followed by removing the solvent via evaporation under reduced pressure at a temperature of 50° C., vacuum drying at a temperature of 40° C., smashing and sieving at 80 meshes, so as to obtain the solid dispersion containing total flavonoids of Desmodium Styracifolium;

c. preparing granules containing total flavonoids of Desmodium Styracifolium: mixing 50 g lactose and 20 g sodium croscarmellose, sieved at 80 meshes in advance respectively, with the solid dispersion containing the total flavonoids of Desmodium Styracifolium obtained in step (b) to be uniform, followed by preparing a soften material with suitable amount of water, granulating at 20 meshes, drying at a temperature of 55° C., size stabilizing, and mixing with 5 g sodium stearyl fumarate to be uniform, so as to obtain the granules containing total flavonoids of Desmodium Styracifolium;

d. capsulizing: capsulizing the granules containing total flavonoids of Desmodium Styracifolium with an encapsulating machine to obtain a capsule containing total flavonoids of Desmodium Styracifolium with a dissolution rate measured to be 91.1%.

### Embodiment 38

### Preparation of Capsules Containing Containing Total Flavonoids of Desmodium Styracifolium

Formula:

| | | |
|---|---|---|
| Total flavonoids of Desmodium Styracifolium | 33 | g |
| Povidone K₃₀ | 132 | g |
| Poloxamer 188 | 66 | g |
| Sodium dodecyl sulfate | 6.6 | g |
| Lactose | 50 | g |
| Cross-linked povidone | 20 | g |
| Aerosil | 1 | g |
| Total | 1000 | capsules |

Preparation: identical to Embodiment 37.

### Embodiment 39

### Preparation of Capsules Containing a Capsule Containing Total Flavonoids of Desmodium Styracifolium

Formula:

| | | |
|---|---|---|
| Total flavonoids of Desmodium Styracifolium | 33 | g |
| Povidone K₃₀ | 132 | g |
| Poloxamer 188 | 66 | g |
| Sodium dodecyl sulfate | 19.8 | g |
| Lactose | 30 | g |
| Sodium croscarmellose | 20 | g |
| Sodium stearyl fumarate | 3 | g |
| Total | 1000 | capsules |

Preparation: identical to Embodiment 37.

### Embodiment 40

### Preparation of Capsules Containing Containing Total Flavonoids of Desmodium Styracifolium

Formula:

| | | |
|---|---|---|
| Total flavonoids of Desmodium Styracifolium | 33 | g |
| Povidone K₃₀ | 132 | g |
| Poloxamer 188 | 66 | g |
| Sodium dodecyl sulfate | 33 | g |
| Microcrystalline cellulose | 10 | g |
| Sodium croscarmellose | 20 | g |
| Magnesium stearate | 2 | g |
| Total | 1000 | capsules |

Preparation: identical to Embodiment 37.

### Embodiment 41

### Preparation of Capsules Containing Containing Total Flavonoids of Desmodium Styracifolium

Formula:

| | | |
|---|---|---|
| Total flavonoids of Desmodium Styracifolium | 33 | g |
| Povidone K₃₀ | 132 | g |
| Poloxamer 188 | 66 | g |
| Tween 80 | 19.8 | g |
| Lactose | 40 | g |
| Sodium carboxymethyl starch | 5 | g |
| Sodium stearyl fumarate | 4 | g |
| Total | 1000 | capsules |

Preparation: identical to Embodiment 37. Dissolution ratio is determined to be 91.5%.

### Embodiment 42

### Preparation of Capsules Containing Containing Total Flavonoids of Desmodium Styracifolium

Formula:

| | | |
|---|---|---|
| Total flavonoids of Desmodium Styracifolium | 33 | g |
| Povidone K₃₀ | 132 | g |
| Poloxamer 188 | 66 | g |
| Methoxypolyethylene glycol | 19.8 | g |
| Microcrystalline cellulose | 20 | g |
| Sodium carboxymethyl starch | 15 | g |
| Sodium stearyl fumarate | 3 | g |
| Total | 1000 | capsules |

Preparation: identical to Embodiment 37.

### Embodiment 43

### Preparation of Capsules Containing Containing Total Flavonoids of Desmodium Styracifolium

Formula:

| | | |
|---|---|---|
| Total flavonoids of Desmodium Styracifolium | 50 | g |
| Povidone K₃₀ | 200 | g |
| Poloxamer 188 | 100 | g |
| Sodium dodecyl sulfate | 10 | g |
| Lactose | 50 | g |
| Sodium croscarmellose | 20 | g |
| Sodium stearyl fumarate | 5 | g |
| Total | 1000 | capsules |

Preparation: identical to Embodiment 37.

### Embodiment 44

### Preparation of Capsules Containing Containing Total Flavonoids of Desmodium Styracifolium

Formula:

| | | |
|---|---|---|
| Total flavonoids of Desmodium Styracifolium | 50 | g |
| Povidone K₃₀ | 200 | g |
| Poloxamer 188 | 100 | g |
| Sodium dodecyl sulfate | 50 | g |
| Lactose | 50 | g |
| Sodium croscarmellose | 20 | g |
| Sodium stearyl fumarate | 5 | g |
| Total | 1000 | capsules |

Preparation: identical to Embodiment 37.

### Embodiment 45

### Preparation of Capsules Containing Containing Total Flavonoids of Desmodium Styracifolium

Formula:

| | | |
|---|---|---|
| Total flavonoids of Desmodium Styracifolium | 50 | g |
| Povidone K₃₀ | 200 | g |
| Poloxamer 188 | 100 | g |
| Tween 80 | 30 | g |
| Lactose | 50 | g |
| Sodium croscarmellose | 20 | g |
| Sodium stearyl fumarate | 5 | g |
| Total | 1000 | capsules |

Preparation: identical to Embodiment 37.

### Embodiment 46

### Preparation of Capsules Containing Containing Total Flavonoids of Desmodium Styracifolium

Formula:

| | | |
|---|---|---|
| Total flavonoids of Desmodium Styracifolium | 50 | g |
| Povidone K₃₀ | 200 | g |
| Poloxamer 188 | 100 | g |
| Methoxypolyethylene glycol | 30 | g |
| Lactose | 50 | g |
| Sodium croscarmellose | 20 | g |
| Sodium stearyl fumarate | 5 | g |
| Total | 1000 | capsules |

Preparation: identical to Embodiment 37. Dissolution ratio is determined to be 91.0%.

### Embodiment 47

### Preparation of Capsules Containing Containing Total Flavonoids of Desmodium Styracifolium

Formula:

| | | |
|---|---|---|
| Total flavonoids of Desmodium Styracifolium | 50 | g |
| Povidone K₃₀ | 200 | g |
| Poloxamer 188 | 100 | g |
| Sodium dodecyl sulfate | 30 | g |
| Pregelatinized starch | 50 | g |
| Sodium croscarmellose | 20 | g |
| Sodium stearyl fumarate | 5 | g |
| Total | 1000 | capsules |

Preparation: identical to Embodiment 37.

### Embodiment 48

### Preparation of Capsules Containing Containing Total Flavonoids of Desmodium Styracifolium

Formula:

| | | |
|---|---|---|
| Total flavonoids of Desmodium Styracifolium | 50 | g |
| Povidone K₃₀ | 200 | g |
| Poloxamer 188 | 100 | g |
| Sodium dodecyl sulfate | 50 | g |
| Cross-linked povidone | 50 | g |
| Sodium croscarmellose | 20 | g |
| Sodium stearyl fumarate | 5 | g |
| Total | 1000 | capsules |

Preparation: identical to Embodiment 37.

### Embodiment 49

### Preparation of Capsules Containing Containing Total Flavonoids of Desmodium Styracifolium

Formula:

| | | |
|---|---|---|
| Total flavonoids of Desmodium Styracifolium | 50 | g |
| Povidone K₃₀ | 200 | g |
| Poloxamer 188 | 100 | g |
| Methoxypolyethylene glycol | 30 | g |
| Microcrystalline cellulose | 50 | g |
| Sodium croscarmellose | 20 | g |
| Aerosil | 5 | g |
| Total | 1000 | capsules |

Preparation: identical to Embodiment 37.

### Embodiment 50

### Preparation of Capsules Containing Containing Total Flavonoids of Desmodium Styracifolium

Formula:

| | | |
|---|---|---|
| Total flavonoids of Desmodium Styracifolium | 66.5 | g |
| Povidone K₃₀ | 266 | g |
| Poloxamer 188 | 133 | g |
| Sodium dodecyl sulfate | 13.3 | g |
| Microcrystalline cellulose | 20 | g |
| Sodium carboxymethyl starch | 10 | g |
| Sodium stearyl fumarate | 8 | g |
| Total | 1000 | capsules |

Preparation: identical to Embodiment 37.

### Embodiment 51

### Preparation of Capsules Containing Containing Total Flavonoids of Desmodium Styracifolium

Formula:

| | | |
|---|---|---|
| Total flavonoids of Desmodium Styracifolium | 66.5 | g |
| Povidone K₃₀ | 266 | g |
| Poloxamer 188 | 133 | g |
| Sodium dodecyl sulfate | 39.9 | g |
| Lactose | 10 | g |
| Sodium croscarmellose | 15 | g |
| Sodium stearyl fumarate | 6 | g |
| Total | 1000 | capsules |

Preparation: identical to Embodiment 37. Dissolution ratio is determined to be 91.3%.

### Embodiment 52

### Preparation of Capsules Containing Containing Total Flavonoids of Desmodium Styracifolium

Formula:

| | | |
|---|---|---|
| Total flavonoids of Desmodium Styracifolium | 66.5 | g |
| Povidone K₃₀ | 266 | g |
| Poloxamer 188 | 133 | g |
| Sodium dodecyl sulfate | 66.5 | g |
| Lactose | 1 | g |
| Cross-linked povidone | 10 | g |
| Magnesium stearate | 10 | g |
| Total | 1000 | capsules |

Preparation: identical to Embodiment 37.

### Embodiment 53

### Preparation of Capsules Containing Containing Total Flavonoids of Desmodium Styracifolium

Formula:

| | | |
|---|---|---|
| Total flavonoids of Desmodium Styracifolium | 66.5 | g |
| Povidone K₃₀ | 266 | g |
| Poloxamer 188 | 133 | g |
| Tween 80 | 39.9 | g |
| Lactose | 10 | g |
| Sodium croscarmellose | 50 | g |
| Sodium stearyl fumarate | 1 | g |
| Total | 1000 | capsules |

Preparation: identical to Embodiment 37.

### Embodiment 54

### Preparation of Capsules Containing Containing Total Flavonoids of Desmodium Styracifolium

Formula:

| | | |
|---|---|---|
| Total flavonoids of Desmodium Styracifolium | 66.5 | g |
| Povidone K₃₀ | 266 | g |
| Poloxamer 188 | 133 | g |
| Methoxypolyethylene glycol | 39.9 | g |
| Pregelatinized starch | 15 | g |
| Sodium croscarmellose | 30 | g |
| Sodium stearyl fumarate | 5 | g |
| Total | 1000 | capsules |

Preparation: identical to Embodiment 37.

### Embodiment 55

### Preparation of Capsules Containing Containing Total Flavonoids of Desmodium Styracifolium

Formula:

| | | |
|---|---|---|
| Total flavonoids of | 100 | g |
| Desmodium Styracifolium | | |
| Povidone K₃₀ | 400 | g |
| Poloxamer 188 | 200 | g |
| Sodium dodecyl sulfate | 20 | g |
| Pregelatinized starch | 50 | g |
| Sodium croscarmellose | 20 | g |
| Sodium stearyl fumarate | 5 | g |
| Total | 1000 | capsules |

Preparation: identical to Embodiment 37.

### Embodiment 56

### Preparation of Capsules Containing Containing Total Flavonoids of Desmodium Styracifolium

Formula:

| | | |
|---|---|---|
| Total flavonoids of | 100 | g |
| Desmodium Styracifolium | | |
| Povidone K₃₀ | 400 | g |
| Poloxamer 188 | 200 | g |
| Sodium dodecyl sulfate | 60 | g |
| Lactose | 50 | g |
| Cross-linked povidone | 40 | g |
| Sodium stearyl fumarate | 8 | g |
| Total | 1000 | capsules |

Preparation: identical to Embodiment 37.

### Embodiment 57

### Preparation of Capsules Containing Containing Total Flavonoids of Desmodium Styracifolium

Formula:

| | | |
|---|---|---|
| Total flavonoids of | 100 | g |
| Desmodium Styracifolium | | |
| Povidone K₃₀ | 400 | g |
| Poloxamer 188 | 200 | g |
| Sodium dodecyl sulfate | 100 | g |
| Microcrystalline cellulose | 10 | g |
| Sodium carboxymethyl starch | 20 | g |
| Sodium stearyl fumarate | 10 | g |
| Total | 1000 | capsules |

Preparation: identical to Embodiment 37.

### Embodiment 58

### Preparation of Capsules Containing Containing Total Flavonoids of Desmodium Styracifolium

Formula:

| | | |
|---|---|---|
| Total flavonoids of | 100 | g |
| Desmodium Styracifolium | | |
| Povidone K₃₀ | 400 | g |
| Poloxamer 188 | 200 | g |
| Tween 80 | 60 | g |
| Lactose | 10 | g |
| Sodium croscarmellose | 15 | g |
| Sodium stearyl fumarate | 8 | g |
| Total | 1000 | capsules |

Preparation: identical to Embodiment 37.

### Embodiment 59

### Preparation of Capsules Containing Containing Total Flavonoids of Desmodium Styracifolium

Formula:

| | | |
|---|---|---|
| Total flavonoids of | 100 | g |
| Desmodium Styracifolium | | |
| Povidone K₃₀ | 400 | g |
| Poloxamer 188 | 200 | g |
| Methoxypolyethylene glycol | 60 | g |
| Lactose | 15 | g |
| Sodium croscarmellose | 10 | g |
| Sodium stearyl fumarate | 8 | g |
| Total | 1000 | capsules |

Preparation: identical to Embodiment 37.

### Embodiment 60

### Preparation of Capsules Containing Containing Total Flavonoids of Desmodium Styracifolium

Formula:

| | | |
|---|---|---|
| Total flavonoids of | 133 | g |
| Desmodium Styracifolium | | |
| Povidone K₃₀ | 532 | g |
| Poloxamer 188 | 266 | g |
| Sodium dodecyl sulfate | 26.6 | g |
| Lactose | 20 | g |
| Sodium croscarmellose | 20 | g |
| Sodium stearyl fumarate | 8 | g |
| Total | 1000 | capsules |

Preparation: identical to Embodiment 37.

### Embodiment 61

### Preparation of Capsules Containing Containing Total Flavonoids of Desmodium Styracifolium

Formula:

| | | |
|---|---|---|
| Total flavonoids of | 133 | g |
| Desmodium Styracifolium | | |
| Povidone K₃₀ | 532 | g |
| Poloxamer 188 | 266 | g |
| Sodium dodecyl sulfate | 79.8 | g |
| Lactose | 20 | g |
| Cross-linked povidone | 25 | g |
| Sodium stearyl fumarate | 9 | g |
| Total | 1000 | capsules |

Preparation: identical to Embodiment 37.

### Embodiment 62

### Preparation of Capsules Containing Containing Total Flavonoids of Desmodium Styracifolium

Formula:

| | | |
|---|---|---|
| Total flavonoids of | 133 | g |
| Desmodium Styracifolium | | |
| Povidone K₃₀ | 532 | g |
| Poloxamer 188 | 266 | g |
| Sodium dodecyl sulfate | 133 | g |
| Microcrystalline cellulose | 1 | g |
| Sodium carboxymethyl starch | 1 | g |
| Sodium stearyl fumarate | 10 | g |
| Total | 1000 | capsules |

Preparation: identical to Embodiment 37.

### Embodiment 63

### Preparation of Capsules Containing Containing Total Flavonoids of Desmodium Styracifolium

Formula:

| | | |
|---|---|---|
| Total flavonoids of | 133 | g |
| Desmodium Styracifolium | | |
| Povidone K₃₀ | 532 | g |
| Poloxamer 188 | 266 | g |
| Tween 80 | 79.8 | g |
| Lactose | 10 | g |
| Sodium croscarmellose | 10 | g |
| Magnesium stearate | 8 | g |
| Total | 1000 | capsules |

Preparation: identical to Embodiment 37.

### Embodiment 64

### Preparation of Capsules Containing Containing Total Flavonoids of Desmodium Styracifolium

Formula:

| | | |
|---|---|---|
| Total flavonoids of | 133 | g |
| Desmodium Styracifolium | | |
| Povidone K₃₀ | 532 | g |
| Poloxamer 188 | 266 | g |
| Methoxypolyethylene glycol | 79.8 | g |
| Pregelatinized starch | 5 | g |
| Cross-linked povidone | 5 | g |
| Magnesium stearate | 5 | g |
| Total | 1000 | capsules |

Preparation: identical to Embodiment 37.

### Embodiment 65

### General Pharmacological Experiments of Total Flavonoids of Desmodium Styracifolium

Experiment object: to observe the pharmacological effect of total flavonoids of Desmodium Styracifolium on general behaviour, state, central nervous system and digestive system of an animal.

Experiment animals and administration: Kunming mice, female, having a weight ranging from 18 g to 22 g, provided by Animal Center of Academy of Military Medical Science with a permit number of experiment animal quality: SOCK (Military) 2002-001, were raised in a mice experiment room of the center with an experiment proved facility number: SYXK (Military) 2002-001.

Experiment grouping: all mice were randomly divided into four groups, i.e., a reference group (administrated with 0.5% sodium carboxymethyl cellulose via gavage), a low-dosage group of total flavonoids of Desmodium Styracifolium (75 mg/kg), a middle-dosage group of total flavonoids of Desmodium Styracifolium (150 mg/kg) and a high-dosage group of total flavonoids of Desmodium Styracifolium (300 mg/kg). Each group contained 10 to 20 mice.

Single gavage was chosen to be the administration route with an administration volume of 0.6 ml/mouse.

Indicators and Results:

1.1 Effects of Total Flavonoids of Desmodium Styracifolium on General Behaviour of Mouse

The general behaviour of mice was observed according to Bastian classification. Each group contained 10 mice, and the observation started after 15 min from gavage for continuous 60 min, which was performed once again after 24 hours. The observation was made to mental, gait, eye, tail, skin, hair and faeces.

After the observation to the general behaviour of the mice, the total flavonoids of Desmodium Styracifolium in the low-, middle- or high-dosage group (75 mg/kg, 150 mg/kg and 300 mg/kg) had little effects on the animal behaviour, action, activity, emotion and gait, with non-significant difference as compared with the reference group.

1.2 Effects of Total Flavonoids of Desmodium Styracifolium on Spontaneous Activity

The results, recorded by a photoelectric method, showed that the spontaneous activities of the mice administrated with different dosages of total flavonoids of Desmodium Styracifolium via gavage had non-significant difference as compared with the reference group, and specific data was shown in Table 9.

TABLE 9
Effect on spontaneous activity of the mice adminitrated
with total flavonoids of Desmodium Styracifolium via gavage

| | | before | | | |
|---|---|---|---|---|---|
| animal | dosage | administration | after administration (times/3 min) | | |
| number | (mg/kg) | (times/3 min) | 30 min | 60 min | 120 min |
| 20 | 0 | 43.5 ± 7.2 | 41.8 ± 3.3 | 40.3 ± 3.1 | 37.8 ± 2.2 |
| 20 | 75 | 43.8 ± 5.0 | 41.3 ± 3.1 | 39.8 ± 3.3 | 38.5 ± 1.3 |
| 20 | 150 | 44.8 ± 5.0 | 39.8 ± 2.2 | 39.3 ± 1.7 | 39.5 ± 1.3 |
| 20 | 300 | 43.5 ± 4.2 | 40.5 ± 1.9 | 39.8 ± 2.2 | 39.5 ± 1.9 |

1.3 Effects of Total Flavonoids of Desmodium Styracifolium on Activating Central Nervous System of Mice

After administrated with total flavonoids of Desmodium Styracifolium via gavage, seizure lasting duration of mouse was observed by subjecting ear tips, applied with an appropriate amount of saline and clamped by a fish-mouth clamp at both sides, to electricity stimulation at a voltage of 110 V for 0.3 second.

As can be seen from obtaining result that the seizure lasting duration caused by the electricity stimulation was not significantly prolonged or shortened by total flavonoids of Desmodium Styracifolium in the low-, middle- or high-dosage group (75 mg/kg, 150 mg/kg and 300 mg/kg) as compared with the reference group; while the seizure occurrence was not changed significantly either (see the specific data shown in Table 10), thus indicating that the total flavonoids had no obvious activating effect on central nervous system via gavage administration.

TABLE 10
Effects on activating central nervous system of the mice administrated
with total flavonoids of Desmodium Styracifolium via gavage

| animal | | dosage | seizure lasting |
|---|---|---|---|
| number | weight (g) | (mg/kg) | duration (sec) |
| 10 | 21.1 ± 0.6 | 0 | 32.8 ± 5.3 |
| 10 | 20.9 ± 0.8 | 75 | 37.1 ± 8.1 |
| 10 | 20.3 ± 0.7 | 150 | 37.7 ± 6.0 |
| 10 | 21.0 ± 0.9 | 300 | 35.5 ± 8.7 |

1.4 Effects of Total Flavonoids of Desmodium Styracifolium on Digestive System of Mice

All mice were randomly grouped such that each group contained 10 mice, all of which were fasten for 12 hours before starting experiment. After 1 hour from administration with total flavonoids of Desmodium Styracifolium, the experiment mice were administrated with a suspending solution made from 5% carbon powder and 10% Arabic gum, with administration volume of 0.2 ml per mouse. All experiment mice were sacrificed after 20 minutes from the administration with the suspending solution for gastrointestinal tract harvest. The gastrointestinal tract was straighten on a glass plate for measuring a distance from pylorus to where the carbon powder headed with a ruler, then a percentage of such the distance to the total length of the gastrointestinal tract was calculated. Obtaining results showed that total flavonoids of Desmodium Styracifolium had no obvious effects on gastrointestinal movement. Specific data was shown in Table 11.

TABLE 11
Effects on propelling rate of the mice administrated with
total flavonoids of Desmodium Styracifolium via gavage

| | total length of | distance from pylorus to where | |
|---|---|---|---|
| dosage | gastrointestinal | the carbon powder headed | propelling |
| (mg/kg) | tract (cm) | (cm) | rate (%) |
| 0 | 52.2 ± 1.9 | 30.2 ± 2.4 | 57.8 ± 4.2 |
| 75 | 52.5 ± 1.7 | 31.6 ± 2.4 | 58.1 ± 3.5 |
| 150 | 52.3 ± 2.3 | 28.8 ± 3.5 | 56.8 ± 5.0 |
| 300 | 52.0 ± 1.9 | 29.8 ± 2.9 | 58.0 ± 3.4 |

### Example 66

### Pharmacodynamic Experiments of Total Flavonoids of Desmodium Styracifolium in Animals

1.1 Experiment of Therapeutic Effects of Total Flavonoids of Desmodium Styracifolium on Ethylene Glycol Calcium Oxalate Kidney Stones in Rats

As compared with the reference group (administrated with 0.5% sodium carboxymethyl cellulose via gavage), the total flavonoids of Desmodium Styracifolium in four dosage groups (50 mg/kg/day, 100 mg/kg/day, 200 mg/kg/day, 400 mg/kg/day) inhibited amount of calcium oxalate crystalline polymer in kidney with a significant dose-effect relationship (P<0.05-0.01); reduced the formation rate of kidney stones (P<0.05-0.01); decreased creatinine content (P<0.05-0.01) and uric acid content (P<0.05-0.01) in serum, and improved kidney function of rats.

1.2 Experiment of Preventing Effects of Total Flavonoids of Desmodium Styracifolium on Ethylene Glycol-Induced Toxic Calcium Oxalate Kidney Stones in Rats

As compared with the reference group (administrated with 0.5% sodium carboxymethyl cellulose via gavage), the total flavonoids of Desmodium Styracifolium in three dosage groups (50 mg/kg/day, 100 mg/kg/day, 200 mg/kg/day) alleviated pyclectasis, reduced the formation rate of kidney stones, decreased the amount of the calcium oxalate crystalline polymer (P<0.01-0.001) and decreased the creatinine content and the uric acid content in serum (P<0.05-0.01).

1.3 Experiment of Dissolving Effect of Total Flavonoids of Desmodium Styracifolium on Implanted Human Bladder Stones in Rats

As compared with the reference group (administrated with 0.5% sodium carboxymethyl cellulose via gavage), total flavonoids of Desmodium Styracifolium in three dosage groups (100 mg/kg/day, 200 mg/kg/day, 400 mg/kg/day) had effects of dissolving stones and reducing the formation of new stones. The total flavonoids of Desmodium Styracifolium in 100 mg/kg/day group lightened the stone weight (P<0.05). The total flavonoids of Desmodium Styracifolium in 200 gm/kg/day group lightened the stone weight (P<0.05) and dissolved 20% stones. The total flavonoids of Desmodium Styracifolium in 400 gm/kg/day group lightened the stone weigh (P<0.01) and dissolved 30% stones.

1.4 Experiment of Diuretic Effects of Total Flavonoids of Desmodium Styracifolium on Rats Suffering Ethylene Glycol-Induced Kidney Stones and Normal Rats

As compared with a reference group (administrated with 0.5% sodium carboxymethyl cellulose via gavage), rats in three dosage groups (50 mg/kg/day, 100 mg/kg/day, 200 mg/kg/day) had a total urine output ranging from 76.4 to 89.5 ml, which was more than that of rats in the normal group (48.1 ml) by 29-36 ml after 6 hours from single administration. After 4 weeks treatment with administration to those rats suffering stones, the urine output within 12 hours was increased significantly, more than that in the model group by 12-36%.

1.5 Experiment of Inhibition Effects of Total Flavonoids of Desmodium Styracifolium on Swelling Degree and Rate Swelling in Rat Toe Injected with Fresh Albumen

As compared with the reference group (administrated with 0.5% sodium carboxymethyl cellulose via gavage), total flavonoids of Desmodium Styracifolium in three dosage groups (100 mg/kg/day, 200 mg/kg/day, 400 mg/kg/day) alleviated the swelling degree and the rate swelling in rat toe injected with fresh albumen, indicating that total flavonoids of Desmodium Styracifolium has a certain anti-inflammatory effect and has an obvious inhibiting effect on proliferation of granulation tissue.

### Example 67

### Acute Toxicity Test of Total Flavonoids of Desmodium Styracifolium in Animals

1.1 Acute Toxicity Tests of Total Flavonoids of Desmodium Styracifolium in Mice

All mice were randomly divided into 6 groups, each containing 20 mice with 10 male and 10 female, with 0.85 distances between groups. After administration, decreased activities, unstable gait, weakened breaths appeared in animals. Most mice died within an hour after the administration, and a few of mice died within 1 to 6 hours after the administration. After calculation by Bliss, LD50 for female was 18.162 g/kg with an upper limit of 20.199 g/kg and a lower limit of 16.326 g/kg under a confidence limit of 95%; LD50 for male was 17.084 g/kg with an upper limit of 18.975 g/kg and a lower limit of 15.301 g/kg under a confidence limit of 95%, with no obvious difference as for LD50 between female and male. According to the results described above, total flavonoids of Desmodium Styracifolium could be recognized as a substantially nontoxic medicament.

1.2 Acute Toxicity Tests of Total Flavonoids of Desmodium Styracifolium in Rats

The test was performed according to “fixed dosage by single oral administration”. Rats were administrated with 2000 mg/kg total flavonoids of Desmodium Styracifolium for preliminary tests, resulting in nonobvious acute toxic reaction; accordingly, 2000 mg/kg was taken as the fixed dosage for formal tests.

Rats for the test were randomly divided into a reference group and an administration group, each containing 10 animals with 5 female and 5 male. Rats in the administration group were administrated with 2000 mg/kg total flavonoids of Desmodium Styracifolium by single gavage, with an administration volume of 2.0 ml/100 g body weight. Rats in the reference group were administrated with 0.5% sodium carboxymethyl cellulose by single gavage, with an administration volume of 2.0 ml/100 g body weight.

Rats in the administration group became lazy to move within 3 hours from the administration; excreted faeces in an ash black color after 1 day from the administration; consumed slightly reduced amount of food, had mildly inhibited increasement in body weight, which recovered to those in the reference group. According to the results described above, total flavonoids of Desmodium Styracifolium could be regarded as a tested medicament without severe and acute toxicity.

### Embodiment 68

In this embodiment, tablets containing total flavonoids of Desmodium Styracifolium prepared by a common wet granulation process without solid dispersion carrier material were used as a control to compare with that prepared by the present method.

The formula (formula 1) used in comparative embodiment was as follows:

| Total flavonoids of | 50 | g |
|---|---|---|
| Desmodium Styracifolium | | |
| Lactose | 50 | g |
| Sodium croscarmellose | 20 | g |
| Sodium stearyl fumarate | 1 | g |
| Water | suitable amount | |
| Total | 1000 | tablets |

Preparation method of the comparative embodiment was performed by the following steps:

a. preparing the total flavonoids of Desmodium Styracifolium according to Embodiment 8;

b. sieving 50 g total flavonoids of Desmodium Styracifolium, 50 g lactose, and 20 g sodium croscarmellose in respective formula dosage at 80 meshes, respectively;

c. evenly mixing total flavonoids of Desmodium Styracifolium with lactose and sodium croscarmellose and, followed by preparing a soften material with suitable amount of water, granulating at 20 meshes, drying at a temperature of 55° C., size stabilizing, uniformly mixing with sodium stearyl fumarate, and tableting, to obtain a tablet containing total flavonoids of Desmodium Styracifolium.

Method for determining the dissolution rate:

Dissolution rate was measured in accordance with the First Method in appendix XC, part II, Chinese Pharmacopoeia, 2010 edition.

Testing sample solutions were obtained by the following steps: dissolving the above-obtained tablet in 1000 ml water (as a dissolution medium) contained in a beaker; setting a rotation rate of dissolution rate analysis instruments to be 100 rpm per min; taking 10 ml from the beaker after 5, 15, 25, 35, 45 and 60 min from operations specified in the First Method, followed by filtration; taking 1 ml secondary filtrate precisely each into a 5 ml volumetric flask and adding 0.1 M hydrochloric acid up to graduation on the volumetric flask, followed by shaken to be uniform, thereby obtaining the testing sample solutions.

Reference sample solutions were obtained by the following steps: weighing a certain amount of schaftosides precisely as a reference sample; dissolving the reference sample with an appropriate volume of ethanol in a volumetric flask; adding 0.1 M hydrochloric acid up to graduation on the volumetric flask, thereby obtaining the reference sample solutions each having a schaftoside concentration of 15 μg/ml.

The testing sample solutions and the reference sample solutions were subjected to ultraviolet spectrophotometry (appendix IV A) at 270 nm, for calculating the dissolution rate of each tablet and the dissolution rate of each tablet was calculated. The dissolution rate was measured to be 75.0%.

Through researching tests, the solid dispersion containing total flavonoids of Desmodium Styracifolium was obtained to use as follow: dissolving the total flavonoids of Desmodium Styracifolium and povidone K₃₀ used as a hydrophilic carrier of solid dispersion with a weight of 1:5 to 12, with 50% ethanol under stirring and at a temperature of 65° C., followed by removing the solvent via evaporation under reduced pressure at a temperature of 50° C., vacuum drying at a temperature of 50° C., smashing and sieving at 80 meshes.

Screening on Formula 2 to Formula 7 with povidone K₃₀ used as solid dispersion carrier are shown in Table 12.

Preparation process was performed by the following steps: mixing lactose and sodium croscarmellose with the solid dispersion containing total flavonoids of Desmodium Styracifolium, sieved at 80 meshes in advance, respectively, to be uniform, followed by preparing a soften material with suitable amount of water, granulating at 20 meshes, drying at a temperature of 55° C., size stabilizing, and mixing with sodium stearyl fumarate to be uniform, then tableting, so as to obtain tablets.

The dissolution rates of total flavonoids of Desmodium Styracifolium were determined, and the determination results are shown in Table 12.

Table 12 screening on Formula of the comparative embodiment and Formulas of the present disclosure

| Formula No. | Formula 1 | Formula 2 | Formula 3 | Formula 4 | Formula 5 | Formula 6 | Formula 7 |
|---|---|---|---|---|---|---|---|
| total flavonoids of | 50 g | / | / | / | / | / | / |
| Desmodium Styracifolium | | | | | | | |
| solid dispersion: total | / | 300 g | 0 | / | / | / | / |
| flavonoids of Desmodium | | | | | | | |
| Styracifolium:povidone | | | | | | | |
| K₃₀ (1:5) | | | | | | | |
| solid dispersion: total | / | / | 325 g | / | / | / | / |
| flavonoids of Desmodium | | | | | | | |
| Styracifolium:povidone | | | | | | | |
| K₃₀ (1:5.5) | | | | | | | |
| solid dispersion: total | / | / | / | 350 g | / | / | / |
| flavonoids of Desmodium | | | | | | | |
| Styracifolium:povidone | | | | | | | |
| K₃₀ (1:6) | | | | | | | |
| solid dispersion: total | / | / | / | / | 400 g | / | / |
| flavonoids of Desmodium | | | | | | | |
| Styracifolium:povidone | | | | | | | |
| K₃₀ (1:7) | | | | | | | |
| solid dispersion: total | / | / | / | / | / | 450 g | / |
| flavonoids of Desmodium | | | | | | | |
| solid dispersion: | | | | | | | |
| Styracifolium:povidone | | | | | | | |
| K₃₀ (1:8) | | | | | | | |
| solid dispersion: total | / | / | / | / | / | / | 650 g |
| flavonoids of Desmodium | | | | | | | |
| Styracifolium:povidone | | | | | | | |
| K₃₀ (1:12) | | | | | | | |
| lactose | 50 | 50 | 50 | 50 | 50 | 50 | 50 |
| sodium croscarmellose | 20 | 20 | 20 | 20 | 20 | 20 | 20 |
| sodium stearyl fumarate | 1 | 5 | 5 | 5 | 5 | 5 | 5 |
| water | suitable | suitable | suitable | suitable | suitable | suitable | suitable |
| | amount | amount | amount | amount | amount | amount | amount |
| determination result of | 75.0 | 79.3 | 79.9 | 82.0 | 79.1 | 79.3 | 78.8 |
| dissolution rate (%) | | | | | | | |

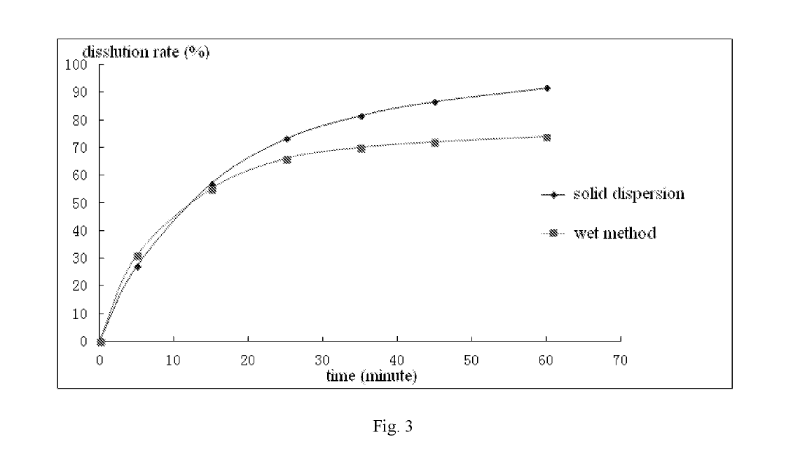

As can be seen from the experimental results shown in Table 12: the dissolution rate of the total flavonoids of Desmodium Styracifolium can be significantly increased by adding povidone K₃₀ with the solid dispersion technology, and the dissolution rate of a tablet prepared according to Formula 4 with a weight ratio of total flavonoids of Desmodium Styracifolium: povidone K₃₀ being 1:6 is higher than that of other tablets under the same testing condition. It has been found from the testing results that, although the dissolution rate has been improved to a certain degree, it still needs to be further improved by optimizing the Formula, so screening is further preformed by adding a certain amount of poloxamer 188 on above basis, i.e. the weight ratio of total flavonoids of Desmodium Styracifolium: povidone K₃₀ is 1:6. FIG. 3 shows a curve for comparing the dissolutions in vitro of a table containing total flavonoids of Desmodium Styracifolium prepared with a solid dispersion process (prepared according to Embodiment 20) and that prepared with a common wet granulation process (Formula 1) according to a comparative embodiment.

Through researching tests, hydrophilic carriers of different solid dispersions, such as povidone K₃₀, poloxamer 188 and polyethylene glycol 6000, are screened, the solid dispersions containing total flavonoids of Desmodium Styracifolium were obtained to use as follow: dissolving 50 g total flavonoids of Desmodium Styracifolium and hydrophilic carriers of solid dispersions with a weight of 1:6, with 50% ethanol under stirring and at a temperature of 65° C., followed by removing the solvent via evaporation under reduced pressure at a temperature of 50° C., vacuum drying at a temperature of 40° C., smashing and sieving at 80 meshes.

Screening on Formula 8 to Formula 14 with the solid dispersion materials are shown in Table 13.

Preparation process was performed by the following steps: mixing lactose and sodium croscarmellose with the solid dispersion containing total flavonoids of Desmodium Styracifolium, sieved at 80 meshes in advance, respectively, to be uniform, followed by preparing a soften material with suitable amount of water, granulating at 20 meshes, drying at a temperature of 55° C., size stabilizing, and mixing with sodium stearyl fumarate to be uniform, then tableting, so as to obtain tablets.

The dissolution rates of total flavonoids of Desmodium Styracifolium were determined, and the determination results are shown in Table 13.

Table 13 further screening on Formulas of the present disclosure

| | Formula | Formula | Formula | Formula | Formula | Formula | Formula |
|---|---|---|---|---|---|---|---|
| Formula No. | 8 | 9 | 10 | 11 | 12 | 13 | 14 |
| solid dispersion: total | 350 g | / | / | / | / | / | / |
| flavonoids of Desmodium | | | | | | | |
| Styracifolium:poloxamer | | | | | | | |
| 188 (1:6) | | | | | | | |
| solid dispersion: total | / | 350 g | / | / | / | / | / |
| flavonoids of Desmodium | | | | | | | |
| Styracifolium: | | | | | | | |
| polyethylene glycol 6000 | | | | | | | |
| (1:6) | | | | | | | |
| solid dispersion: total | / | / | 350 g | / | / | / | / |
| flavonoids of Desmodium | | | | | | | |
| Styracifolium:povidone | | | | | | | |
| K₃₀:poloxamer 188 (1:5:1) | | | | | | | |
| solid dispersion: total | / | / | / | 350 g | / | / | / |
| flavonoids of Desmodium | | | | | | | |
| Styracifolium:povidone | | | | | | | |
| K₃₀:poloxamer 188 (1:4:2) | | | | | | | |
| solid dispersion: total | / | / | / | / | 350 g | / | / |
| flavonoids of Desmodium | | | | | | | |
| Styracifolium:povidone | | | | | | | |
| K₃₀:poloxamer 188 (1:3:3) | | | | | | | |
| solid dispersion: total | / | / | / | / | / | 350 g | / |
| flavonoids of Desmodium | | | | | | | |
| Styracifolium:povidone | | | | | | | |
| K₃₀:poloxamer 188 (1:2:4) | | | | | | | |
| solid dispersion: total | / | / | / | / | / | / | 350 g |
| flavonoids of Desmodium | | | | | | | |
| Styracifolium:povidone | | | | | | | |
| K₃₀:poloxamer 188 (1:1:5) | | | | | | | |
| lactose | 50 | 50 | 50 | 50 | 50 | 50 | 50 |
| sodium croscarmellose | 20 | 20 | 20 | 20 | 20 | 20 | 20 |
| sodium stearyl fumarate | 5 | 5 | 5 | 5 | 5 | 5 | 5 |
| water | suitable | suitable | suitable | suitable | suitable | suitable | suitable |
| | amount | amount | amount | amount | amount | amount | amount |
| determination result of | 81.3 | 80.1 | 83.6 | 86.9 | 85.3 | 84.0 | 82.9 |
| dissolution rate (%) | | | | | | | |

As can be seen from the experimental results shown in Table 13: the dissolution rate of total flavonoids of Desmodium Styracifolium can be significantly increased with the solid dispersion technology, and the dissolution rate of a tablet prepared according to Formula 11 with a weight ratio of total flavonoids of Desmodium Styracifolium: povidone poloxamer 188 being 1:4:2 is higher than that of other tablets under the same testing condition. It has been found from the testing results that, although the dissolution rate has been improved to a certain degree, it still needs to be further improved by optimizing the Formula, so screening is further preformed by adding a certain amount of surfactant on above basis, i.e. the weight ratio of total flavonoids of Desmodium Styracifolium: povidone K₃₀: poloxamer 188 is 1:4:2.

Screening on Formula 15 to Formula 19 which were performed by adding a suitable amount of surfactant on the basis of the solid dispersion, i.e. adding sodium dodecyl sulfate, Tween 80, and methoxypolyethylene glycol, respectively, with different weight ratios on the basis of original different Formulas, are shown in Table 14.

Preparation process was performed by the following steps: mixing lactose and sodium croscarmellose with the solid dispersion containing total flavonoids of Desmodium Styracifolium, sieved at 80 meshes in advance, respectively, to be uniform, followed by preparing a soften material with suitable amount of water, granulating at 20 meshes, drying at a temperature of 55° C., size stabilizing, and mixing with sodium stearyl fumarate to be uniform, then tableting, so as to obtain tablets.

The dissolution rates of total flavonoids of Desmodium Styracifolium were determined, and the determination results are shown in Table 14.

Table 14 screening on Formulas of the present disclosure with surfactant on the basis of solid dispersion

| | Formula No. | | | | |
|---|---|---|---|---|---|
| | Formula | Formula | Formula | Formula | Formula |
| | 15 | 16 | 17 | 18 | 19 |
| solid dispersion: total flavonoids of | 360 g | 0 | / | / | / |
| Desmodium Styracifolium:povidone | | | | | |
| K₃₀:poloxamer 188:sodium dodecyl sulfate | | | | | |
| (1:4:2:0.2) | | | | | |
| solid dispersion: total flavonoids of | / | 380 g | / | / | / |
| Desmodium Styracifolium:povidone K₃₀:poloxamer | | | | | |
| 188:sodium dodecyl sulfate | | | | | |
| (1:4:2:0.6) | | | | | |
| solid dispersion: total flavonoids of | / | / | 400 g | / | / |
| Desmodium Styracifolium:povidone K₃₀:poloxamer | | | | | |
| 188:sodium dodecyl sulfate | | | | | |
| (1:4:2:1.0) | | | | | |
| solid dispersion: total flavonoids of | / | / | / | 380 g | / |
| Desmodium Styracifolium:povidone K₃₀:poloxamer | | | | | |
| 188:Tween 80 (1:4:2:0.6) | | | | | |
| solid dispersion: total flavonoids of | / | / | / | / | 380 g |
| Desmodium Styracifolium:povidone K₃₀:poloxamer | | | | | |
| 188:methoxypolyethylene glycol | | | | | |
| (1:4:2:0.6) | | | | | |
| lactose | 50 | 50 | 50 | 50 | 50 |
| sodium croscarmellose | 20 | 20 | 20 | 20 | 20 |
| sodium stearyl fumarate | 5 | 5 | 5 | 5 | 5 |
| water | suitable | suitable | suitable | suitable | suitable |
| | amount | amount | amount | amount | amount |
| determination result of dissolution rate (%) | 89.1 | 90.7 | 88.9 | 88.1 | 88.6 |

As can be seen from the experimental results shown in Table 14: all the dissolution rates of Formula 15 to Formula 19 are significantly increased and can reach more than 88% by adding sodium dodecyl sulfate, Tween 80, and methoxypolyethylene glycol, respectively, with different weight ratios on the basis of original different Formulas, and in which the improvement of Formula 16 is most obvious. Therefore, the optimal Formula to prepare the solid dispersion containing total flavonoids of Desmodium Styracifolium is determined to be: total flavonoids of Desmodium Styracifolium: povidone K₃₀: poloxamer 188: sodium dodecyl sulfate is 1:4:2:0.6 (by weight).

Reference throughout this specification to “an embodiment,” “some embodiments,” “one embodiment”, “another example,” “an example,” “a specific example,” or “some examples,” means that a particular feature, structure, material, or characteristic described in connection with the embodiment or example is included in at least one embodiment or example of the present disclosure. Thus, the appearances of the phrases such as “in some embodiments,” “in one embodiment”, “in an embodiment”, “in another example,” “in an example,” “in a specific example,” or “in some examples,” in various places throughout this specification are not necessarily referring to the same embodiment or example of the present disclosure. Furthermore, the particular features, structures, materials, or characteristics may be combined in any suitable manner in one or more embodiments or examples.

Although explanatory embodiments have been shown and described, it would be appreciated by those skilled in the art that the above embodiments cannot be construed to limit the present disclosure, and changes, alternatives, and modifications can be made in the embodiments without departing from spirit, principles and scope of the present disclosure, and the scope of the present disclosure is defined by claims and equivalents thereof.

1-42. (canceled)

43. A method for preparing an oral solid formulation containing total flavonoids of Desmodium Styracifolium, comprising:
heating and refluxing a raw material of Desmodium Styracifolium for extraction, 1 to 3 times with 1 to 3 hours for each time, with ethanol having a concentration ranging from 50% to 95% and a weight ranging from 8 to 14 times as heavy as the raw material of Desmodium styracifolium, to obtain an extracting solutions of Desmodium styracifolium followed by mixing;
concentrating mixed extracting solutions of Desmodium Styracifolium, so as to remove ethanol;
subjecting the mixed extracting solutions of Desmodium Styracifolium after concentrated to adsorption onto a macroporous resin column, so as to obtain alcohol extract of Desmodium Styracifolium;
mixing the alcohol extract of Desmodium Styracifolium with a solid dispersion carrier and a surfactant, and preparing a solid dispersion containing total flavonoids of Desmodium Styracifolium via a solid dispersion technology;
mixing the solid dispersion containing total flavonoids of Desmodium Styracifolium with a filler and a disintegrant followed by preparing a soft material, granulating and size stabilizing, and then mixing with a lubricant, so as to obtain a mixture; and
capsulizing the mixture with an encapsulating machine so as to obtain a capsule containing total flavonoids of Desmodium Styracifolium, or tableting the mixture with a tableting machine so as to obtain a tablet containing total flavonoids of Desmodium Styracifolium.

44-46. (canceled)

47. The method according to claim 43, wherein the solid dispersion carrier is at least one of povidone K₃₀ and poloxamer 188.

48. The method according to claim 43, wherein the surfactant is at least one of Tween 80, methoxypolyethylene glycol, and sodium dodecyl sulfate.

49. The method according to 43, wherein the filler is at least one of microcrystalline cellulose, lactose, and pregelatinized starch.

50. The method according to 43, wherein the disintegrant is at least one of sodium croscarmellose, cross-linked povidone, and sodium carboxymethyl starch.

51. The method according to 43, wherein the lubricant is at least one of magnesium stearate, aerosil, and sodium stearyl fumarate.

52. The method according to claim 43, further comprising:
weighing a raw material of Desmodium Styracifolium, heating and refluxing the raw material for extraction, 1 to 3 times with 1 to 3 hours for each time, at a temperature of 50° C. to 60° C. with ethanol having a concentration ranging from 50% to 95% and a weight ranging from 8 to 14 times as heavy as the raw material, so as to obtain alcohol extracting solutions of Desmodium Styracifolium followed by mixing;
concentrating the alcohol extracting solution to be of a volume 2 to 8 times the weight of the raw material followed by still standing and filtering to obtain a filtrate;
subjecting the filtrate to adsorption onto an AB-8 macroporous resin column at a flow rate ranging from 1 to 3 column bed volumes per hour, eluting and purifying with water having a volume ranging from 8 to 12 times the weight of filled resin, and eluting with ethanol having a concentration of 40% to 95% and a volume ranging from 6 to 10 column bed volumes at a flow rate ranging from 2 to 4 column bed volumes per hour, to obtain an eluted solution; and
concentrating the eluted solution into a concentrated solution with a relative density ranging from 1.10 to 1.30 followed by drying and then smashing, so as to obtain the alcohol extract of Desmodium Styracifolium.

53. The method according to claim 43, further comprising:
dissolving the total flavonoids of Desmodium Styracifolium, a solid dispersion carrier and a surfactant in respective formula dosage, sieved at 40 to 200 meshes in advance respectively, with ethanol having a concentration ranging from 20% to 75% under stirring and at a temperature of 50° C. to 75° C., followed by removing the solvent via evaporation under reduced pressure at a temperature of 30° C. to 75° C., vacuum drying at a temperature of 30° C. to 60° C., smashing and sieving at 40 to 200 meshes, so as to obtain the solid dispersion containing total flavonoids of Desmodium Styracifolium;
mixing a filler and a disintegrant, sieved at 40 to 100 meshes in advance, respectively, with the solid dispersion containing total flavonoids of Desmodium Styracifolium, followed by preparing a soften material, granulating at 10 to 30 meshes, drying at a temperature of 30° C. to 75° C., size stabilizing, and mixing with a lubricant, then capsulizing the mixture obtained with an encapsulating machine so as to obtain the capsule containing total flavonoids of Desmodium Styracifolium.

54. The method according to claim 43, further comprising:
dissolving the total flavonoids of Desmodium Styracifolium, a solid dispersion carrier and a surfactant in respective formula dosage, sieved at 40 to 200 meshes in advance respectively, with ethanol having a concentration ranging from 20% to 75% under stirring and at a temperature of 50° C. to 75° C., followed by removing the solvent via evaporation under reduced pressure at a temperature of 30° C. to 75° C., vacuum drying at a temperature of 30° C. to 60° C., smashing and sieving at 40 to 200 meshes, so as to obtain the solid dispersion containing total flavonoids of Desmodium Styracifolium;
mixing a filler and a disintegrant, sieved at 40 to 100 meshes in advance, respectively, with the solid dispersion containing total flavonoids of Desmodium Styracifolium, followed by preparing a soften material, granulating at 10 to 30 meshes, drying at a temperature of 30° C. to 75° C., size stabilizing, and mixing with a lubricant, then tableting the mixture obtained with a tableting machine so as to obtain the tablet containing total flavonoids of Desmodium Styracifolium.

55. The method according to claim 43, comprising:
a. preparing the total flavonoids of Desmodium Styracifolium: weighing a raw material of Desmodium Styracifolium in a formula dosage, heating and refluxing at a temperature of 55° C. for 2 hours for first extraction with ethanol having a concentration of 80% and a weight 12 times as heavy as the raw material, heating and refluxing at a temperature of 55° C. for 1.5 hours for second extraction with ethanol having a concentration of 80% and a weight 10 times as heavy as the raw material, so as to obtain alcohol extracting solutions of Desmodium Styracifolium followed by mixing;
concentrating the alcohol extracting solution to be of a volume 5 times the weight of the raw material followed by still standing and filtering, to obtain a filtrate;
subjecting the filtrate to adsorption onto an AB-8 macroporous resin column at a flow rate of 2 column bed volumes per hour, eluting and purifying with water having a volume 10 times the weight of filled resin, and eluting with ethanol having a concentration of 60% and a volume of 8 column bed volumes at a flow rate of 2 column bed volumes per hour, to obtain an eluted solution; and
concentrating the eluted solution into a concentrated solution with a relative density of 1.22, followed by drying under reduced pressure at a temperature of 75° C. and then smashing to obtain the total flavonoids of Desmodium Styracifolium;
b. preparing a solid dispersion containing total flavonoids of Desmodium Styracifolium: dissolving the total flavonoids of Desmodium Styracifolium, povidone K₃₀, poloxamer 188 and sodium dodecyl sulfate in respective formula dosage, sieved at 80 meshes in advance respectively, with ethanol having a concentration of 50% under stirring and at a temperature of 65° C., followed by removing the solvent via evaporation under reduced pressure at a temperature of 50° C., vacuum drying at a temperature of 40° C., smashing and sieving at 80 meshes, so as to obtain the solid dispersion containing total flavonoids of Desmodium Styracifolium;
c. preparing granules containing total flavonoids of Desmodium Styracifolium: mixing lactose and sodium croscarmellose, sieved at 80 meshes in advance respectively, with the solid dispersion containing the total flavonoids of Desmodium Styracifolium to be uniform, followed by preparing a soften material, granulating at 20 meshes, drying at a temperature of 55° C., size stabilizing, and mixing with aerosil to be uniform, so as to obtain the granules containing total flavonoids of Desmodium Styracifolium;
d. capsulizing: capsulizing the granules containing total flavonoids of Desmodium Styracifolium with an encapsulating machine so as to obtain the capsule containing total flavonoids of Desmodium Styracifolium.

56. The method according to claim 43, comprising:
a. preparing the total flavonoids of Desmodium Styracifolium: weighing a raw material of Desmodium Styracifolium in a formula dosage, heating and refluxing at a temperature of 55° C. for 2 hours for first extraction with ethanol having a concentration of 80% and a weight 12 times as heavy as the raw material, heating and refluxing at a temperature of 55° C. for 1.5 hours for second extraction with ethanol having a concentration of 80% and a weight 10 times as heavy as the raw material, so as to obtain alcohol extracting solutions of Desmodium Styracifolium followed by mixing;
concentrating the alcohol extracting solution to be of a volume 5 times the weight of the raw material followed by still standing and filtering, to obtain a filtrate;
subjecting the filtrate to adsorption onto an AB-8 macroporous resin column at a flow rate of 2 column bed volumes per hour, eluting and purifying with water having a volume 10 times the weight of filled resin, and eluting with ethanol having a concentration of 60% and a volume of 8 column bed volumes at a flow rate of 2 column bed volumes per hour, to obtain an eluted solution; and
concentrating the eluted solution into a concentrated solution with a relative density of 1.22, followed by drying under reduced pressure at a temperature of 75° C. and then smashing to obtain the total flavonoids of Desmodium Styracifolium;
b. preparing a solid dispersion containing total flavonoids of Desmodium Styracifolium: dissolving the total flavonoids of Desmodium Styracifolium, povidone K₃₀, poloxamer 188 and sodium dodecyl sulfate in respective formula dosage, sieved at 80 meshes in advance respectively, with ethanol having a concentration of 50% under stirring and at a temperature of 65° C., followed by removing the solvent via evaporation under reduced pressure at a temperature of 50° C., vacuum drying at a temperature of 40° C., smashing and sieving at 80 meshes, so as to obtain the solid dispersion containing total flavonoids of Desmodium Styracifolium;
c. preparing granules containing total flavonoids of Desmodium Styracifolium: mixing lactose and sodium croscarmellose, sieved at 80 meshes in advance respectively, with the solid dispersion containing the total flavonoids of Desmodium Styracifolium to be uniform, followed by preparing a soften material, granulating at 20 meshes, drying at a temperature of 55° C., size stabilizing, and mixing with aerosil to be uniform, so as to obtain the granules containing total flavonoids of Desmodium Styracifolium;
d. tableting: tableting the granules containing total flavonoids of Desmodium Styracifolium with a tableting machine to obtain the tablet containing total flavonoids of Desmodium Styracifolium.

57. The method according to claim 43, wherein 33 to 133 weight parts of total flavonoids of Desmodium Styracifolium, 198 to 798 weight parts of the solid dispersion carrier, 6.6 to 133 weight parts of the surfactant, 1 to 50 weight parts of the filler, 1 to 50 weight parts of the disintegrant, and 1 to 10 weight parts of the lubricant are used.

58. The method according to claim 57, wherein a formula is used which is at least one selected from:
33 weight parts of total flavonoids of Desmodium Styracifolium, 132 weight parts of povidone K₃₀, 66 weight parts of poloxamer 188, 6.6 weight parts of sodium dodecyl sulfate, 50 weight parts of lactose, 20 weight parts of cross-linked povidone, and 1 weight part of aerosil;
33 weight parts of total flavonoids of Desmodium Styracifolium, 132 weight parts of povidone K₃₀, 66 weight parts of poloxamer 188, 19.8 weight parts of sodium dodecyl sulfate, 30 weight parts of lactose, 20 weight parts of sodium croscarmellose, and 3 weight parts of sodium stearyl fumarate;
33 weight parts of total flavonoids of Desmodium Styracifolium, 132 weight parts of povidone K₃₀, 66 weight parts of poloxamer 188, 33 weight parts of sodium dodecyl sulfate, 10 weight parts of microcrystalline cellulose, 20 weight parts of sodium croscarmellose, and 2 weight parts of magnesium stearate;
33 weight parts of total flavonoids of Desmodium Styracifolium, 132 weight parts of povidone K₃₀, 66 weight parts of poloxamer 188, 19.8 weight parts of Tween 80, 40 weight parts of lactose, 5 weight parts of sodium carboxymethyl starch, and 4 weight parts of sodium stearyl fumarate;
33 weight parts of total flavonoids of Desmodium Styracifolium, 132 weight parts of povidone K₃₀, 66 weight parts of poloxamer 188, 19.8 weight parts of methoxypolyethylene glycol, 20 weight parts of microcrystalline cellulose, 15 weight parts of sodium carboxymethyl starch, and 3 weight parts of sodium stearyl fumarate;
50 weight parts of total flavonoids of Desmodium Styracifolium, 200 weight parts of povidone K₃₀, 100 weight parts of poloxamer 188, 10 weight parts of sodium dodecyl sulfate, 50 weight parts of lactose, 20 weight parts of sodium croscarmellose, and 5 weight parts of sodium stearyl fumarat;
50 weight parts of total flavonoids of Desmodium Styracifolium, 200 weight parts of povidone K₃₀, 100 weight parts of poloxamer 188, 30 weight parts of sodium dodecyl sulfate, 50 weight parts of lactose, 20 weight parts of sodium croscarmellose, and 5 weight parts of sodium stearyl fumarate;
50 weight parts of total flavonoids of Desmodium Styracifolium, 200 weight parts of povidone K₃₀, 100 weight parts of poloxamer 188, 50 weight parts of sodium dodecyl sulfate, 50 weight parts of lactose, 20 weight parts of sodium croscarmellose, and 5 weight parts of sodium stearyl fumarate;
50 weight parts of total flavonoids of Desmodium Styracifolium, 200 weight parts of povidone K₃₀, 100 weight parts of poloxamer 188, 30 weight parts of Tween 80, 50 weight parts of lactose, 20 weight parts of sodium croscarmellose, and 5 weight parts of sodium stearyl fumarate;
50 weight parts of total flavonoids of Desmodium Styracifolium, 200 weight parts of povidone K₃₀, 100 weight parts of poloxamer 188, 30 weight parts of methoxypolyethylene glycol, 50 weight parts of lactose, 20 weight parts of sodium croscarmellose, and 5 weight parts of sodium stearyl fumarate;
50 weight parts of total flavonoids of Desmodium Styracifolium, 200 weight parts of povidone K₃₀, 100 weight parts of poloxamer 188, 30 weight parts of sodium dodecyl sulfate, 50 weight parts of pregelatinized starch, 20 weight parts of sodium croscarmellose, and 5 weight parts of sodium stearyl fumarate;
50 weight parts of total flavonoids of Desmodium Styracifolium, 200 weight parts of povidone K₃₀, 100 weight parts of poloxamer 188, 50 weight parts of sodium dodecyl sulfate, 50 weight parts of cross-linked povidone, 20 weight parts of sodium croscarmellose, and 5 weight parts of sodium stearyl fumarate;
50 weight parts of total flavonoids of Desmodium Styracifolium, 200 weight parts of povidone K₃₀, 100 weight parts of poloxamer 188, 30 weight parts of methoxypolyethylene glycol, 50 weight parts of microcrystalline cellulose, 20 weight parts of sodium croscarmellose, and 5 weight parts of aerosol;
66.5 weight parts of total flavonoids of Desmodium Styracifolium, 266 weight parts of povidone K₃₀, 133 weight parts of poloxamer 188, 13.3 weight parts of sodium dodecyl sulfate, 20 weight parts of microcrystalline cellulose, 10 weight parts of sodium carboxymethyl starch, and 8 weight parts of sodium stearyl fumarate;
66.5 weight parts of total flavonoids of Desmodium Styracifolium, 266 weight parts of povidone K₃₀, 133 weight parts of poloxamer 188, 39.9 weight parts of sodium dodecyl sulfate, 10 weight parts of lactose, 15 weight parts of sodium croscarmellose, and 6 weight parts of sodium stearyl fumarate;
66.5 weight parts of total flavonoids of Desmodium Styracifolium, 266 weight parts of povidone K₃₀, 133 weight parts of poloxamer 188, 66.5 weight parts of sodium dodecyl sulfate, 1 weight part of lactose, 10 weight parts of cross-linked povidone, and 10 weight parts of magnesium stearate;
66.5 weight parts of total flavonoids of Desmodium Styracifolium, 266 weight parts of povidone K₃₀, 133 weight parts of poloxamer 188, 39.9 weight parts of Tween 80, 10 weight parts of lactose, 50 weight parts of sodium croscarmellose, and 1 weight part of sodium stearyl fumarate;
66.5 weight parts of total flavonoids of Desmodium Styracifolium, 266 weight parts of povidone K₃₀, 133 weight parts of poloxamer 188, 39.9 weight parts of methoxypolyethylene glycol, 15 weight parts of pregelatinized starch, 30 weight parts of sodium croscarmellose, and 5 weight parts of sodium stearyl fumarate;
100 weight parts of total flavonoids of Desmodium Styracifolium, 400 weight parts of povidone K₃₀, 200 weight parts of poloxamer 188, 20 weight parts of sodium dodecyl sulfate, 50 weight parts of pregelatinized starch, 20 weight parts of sodium croscarmellose, and 5 weight parts of sodium stearyl fumarate;
100 weight parts of total flavonoids of Desmodium Styracifolium, 400 weight parts of povidone K₃₀, 200 weight parts of poloxamer 188, 60 weight parts of sodium dodecyl sulfate, 50 weight parts of lactose, 40 weight parts of cross-linked povidone, and 8 weight parts of sodium stearyl fumarate;
100 weight parts of total flavonoids of Desmodium Styracifolium, 400 weight parts of povidone K₃₀, 200 weight parts of poloxamer 188, 100 weight parts of sodium dodecyl sulfate, 10 weight parts of microcrystalline cellulose, 20 weight parts of sodium carboxymethyl starch, and 10 weight parts of sodium stearyl fumarate;
100 weight parts of total flavonoids of Desmodium Styracifolium, 400 weight parts of povidone K₃₀, 200 weight parts of poloxamer 188, 60 weight parts of Tween 80, 10 weight parts of lactose, 15 weight parts of sodium croscarmellose, and 8 weight parts of sodium stearyl fumarate;
100 weight parts of total flavonoids of Desmodium Styracifolium, 400 weight parts of povidone K₃₀, 200 weight parts of poloxamer 188, 60 weight parts of methoxypolyethylene glycol, 15 weight parts of lactose, 10 weight parts of sodium croscarmellose, and 8 weight parts of sodium stearyl fumarate;
133 weight parts of total flavonoids of Desmodium Styracifolium, 532 weight parts of povidone K₃₀, 266 weight parts of poloxamer 188, 26.6 weight parts of sodium dodecyl sulfate, 20 weight parts of lactose, 20 weight parts of sodium croscarmellose, and 8 weight parts of sodium stearyl fumarate;
133 weight parts of total flavonoids of Desmodium Styracifolium, 532 weight parts of povidone K₃₀, 266 weight parts of poloxamer 188, 79.8 weight parts of sodium dodecyl sulfate, 20 weight parts of lactose, 25 weight parts of cross-linked povidone, and 9 weight parts of sodium stearyl fumarate;
133 weight parts of total flavonoids of Desmodium Styracifolium, 532 weight parts of povidone K₃₀, 266 weight parts of poloxamer 188, 133 weight parts of sodium dodecyl sulfate, 1 weight part of microcrystalline cellulose, 1 weight part of sodium carboxymethyl starch, and 10 weight parts of sodium stearyl fumarate;
133 weight parts of total flavonoids of Desmodium Styracifolium, 532 weight parts of povidone K₃₀, 266 weight parts of poloxamer 188, 79.8 weight parts of Tween 80, 10 weight parts of lactose, 10 weight parts of sodium croscarmellose, and 8 weight parts of magnesium stearate;
133 weight parts of total flavonoids of Desmodium Styracifolium, 532 weight parts of povidone K₃₀, 266 weight parts of poloxamer 188, 79.8 weight parts of methoxypolyethylene glycol, 5 weight parts of pregelatinized starch, 5 weight parts of cross-linked povidone, and 5 weight parts of magnesium stearate;

59-85. (canceled)

86. A solid dispersion formulation containing total flavonoids of Desmodium Styracifolium prepared by the method according to claim 43.

87. A method for treating urinary stone, comprising administrating a solid dispersion formulation containing total flavonoids of Desmodium Styracifolium prepared by the method according to claim 43 to a patient in need thereof.
